(19) United States
(12) Patent Application Publication
(10) Pub. No.: US 2025/0235100 A1
(43) Pub. Date: Jul. 24, 2025

(54) GLUCOSE SENSOR FOR GLYCEMIC ASSESSMENT

(71) Applicant: Abbott Diabetes Care Inc., Alameda, CA (US)

(72) Inventors: Gary A. HAYTER, Oakland, CA (US); Marc B. TAUB, Los Altos, CA (US); Xuandong HUA, Oakland, CA (US); Jean-Pierre COLE, Lynden, WA (US)

(21) Appl. No.: 19/033,063

(22) Filed: Jan. 21, 2025

Related U.S. Application Data
(60) Provisional application No. 63/623,369, filed on Jan. 22, 2024.

(51) Int. Cl. A61B 5/00; A61B 5/145; G16H 10/60; G16H 15/00

(52) U.S. Cl. CPC A61B 5/0004; A61B 5/002; A61B 5/14532; G16H 10/60; G16H 15/00; A61B 2562/08

(57) ABSTRACT

An analyte assessment system includes an analyte sensor, a remote device, and a software application. The remote device is configured to retrieve sensor data of a patient from the analyte sensor. The software application is configured to receive an order for analyte assessment associated with an electronic medical record (EMR) of the patient, generate or retrieve a unique identifier associated with the order, authenticate the unique identifier with the remote device, activate the analyte sensor based on the unique identifier, retrieve sensor data from the remote device, generate a report, and upload the report to the EMR of the patient. Advantageously the system can streamline requests and delivery of analyte reports for third parties, reduce in-person patient interactions and time delays, reduce third party effort, increase security and integrity of patient data, update an EMR of the patient, and operate in a blinded mode for unbiased patient analyte assessment.

### CROSS-REFERENCE TO RELATED APPLICATIONS

This application claims the benefit of U.S. Provisional Application No. 63/623,369, filed Jan. 22, 2024, which is incorporated herein by reference in its entirety.

### FIELD

The present disclosure relates to analyte assessment apparatuses, systems, and methods, for example, software application apparatuses, systems, and methods for requesting, generating, and delivering reports to third parties for assessment of a patient.

### BACKGROUND

The detection and/or monitoring of glucose levels can be vitally important to the health of an individual having diabetes. People with diabetes (PwD) are generally required to monitor their glucose levels to ensure they are maintained within a clinically safe range, and may also use this information to determine if and/or when insulin is needed to reduce glucose levels in their bodies or when additional glucose is needed to raise glucose levels in their bodies. A number of systems allow individuals to monitor their blood glucose, for example, continuous glucose monitoring (CGM). Some of these systems include electrochemical biosensors, including those that use a glucose sensor adapted to be positioned in vivo, for example, with complete or partial insertion into a subcutaneous or transcutaneous site, within the body for continuous in vivo monitoring of glucose levels from bodily fluids of the site.

In some cases, a glucose sensor is applied and activated for a patient by a third party, for example, a clinician. The patient must then wait a period of time (e.g., an hour) in the clinic after application and activation of the glucose sensor so that the clinician can confirm the glucose sensor is functional and working properly. The clinician activates the new glucose sensor and tests its functionality with a special reader maintained at the clinic. The patient must then return to the clinic after an expiration period of the glucose sensor (e.g., 7 days), with the expired glucose sensor, so that the clinician can scan the expired glucose sensor with the special reader and retrieve the sensor data. The clinician must then upload the sensor data to a separate program or server to generate glucose reports for glycemic assessment of the patient.

The current procedure for initiating the glucose assessment and viewing the resulting report is arduous and time-consuming for both patients and third parties, including clinicians, primary care physicians (PCPs), health care professionals (HCPs), and/or caregivers. The patient must be present (e.g., colocated) with the third party for multiple interactions on separate occasions. Further, patients that can access their sensor data (e.g., unblinded mode) may alter their behavior to correct out-of-range glucose values, which can distort (e.g., bias) the glucose reports and prevent the third party (e.g., PCP, HCP, clinician, etc.) from properly understanding the patient's glycemic condition when the patient is not using the glucose sensor.

### SUMMARY

Accordingly, there is a need to develop an analyte assessment system (e.g., glycemic assessment system) that can streamline requests and delivery of analyte reports (e.g., glucose reports) for third parties (e.g., PCPs, HCPs, clinicians, etc.), reduce in-person patient interactions and time delays (e.g., at clinics, lab test facilities, etc.), reduce third party effort, increase security and integrity of patient data, update an electronic medical records (EMR) of the patient (e.g., similar to lab test results), and operate in a blinded mode for unbiased patient analyte assessment. Further, there is a need to develop an analyte assessment system (e.g., glycemic assessment system) that can initiate a clinical trial, create an independent storage server (e.g., cloud server) associated with the clinical trial, export sensor data from multiple participants to the independent storage server, streamline requests and delivery of reports (e.g., glucose reports, participant data, clinical trial metrics, etc.), and provide role-based access control for third parties (e.g., PCPs, HCPs, clinicians, research sites, institutions, industry sponsors, contract research organizations (CROs), etc.) to review participant data, assess therapy changes, monitor participant health and safety, and/or modify parameters of the clinical trial.

In some aspects, a system can include an analyte measurement system, a remote device in communication with the analyte measurement system, and a processor in communication with the remote device. In some aspects, the analyte measurement system can include an analyte sensor (e.g., a glucose sensor). In some aspects, the analyte measurement system can be configured to measure an analyte of a patient. In some aspects, the analyte measurement system can be a glycemic measurement system configured to measure glucose of a patient. In some aspects, the remote device can be configured to receive or retrieve sensor data from the analyte sensor. In some aspects, the processor can be coupled to a memory storing instructions that when executed cause the processor to: receive and store an order for analyte assessment associated with an EMR account of the patient; generate or retrieve a unique identifier associated with the order; transfer the unique identifier to the remote device; activate the analyte measurement system with the remote device and store the unique identifier on the analyte sensor; retrieve and store sensor data and the associated unique identifier from the analyte sensor; generate a report based on the sensor data; and upload the report to the EMR account associated with the unique identifier. Advantageously the system can streamline requests and delivery of analyte reports (e.g., glucose reports) for third parties (e.g., PCPs, HCPs, clinicians, etc.), reduce in-person patient interactions and time delays (e.g., at clinics, lab test facilities, etc.), reduce third party effort, increase security and integrity of patient data, and update an EMR of the patient (e.g., similar to lab test results).

In some aspects, the processor can be configured to receive and store an order for analyte assessment associated with an EMR account of the patient. In some aspects, the processor can be configured to generate or retrieve a unique identifier associated with the order and the EMR account. In some aspects, the processor can be configured to transfer the unique identifier to the remote device. In some aspects, the processor can be configured to activate the analyte measurement system with the remote device and store the unique identifier on the analyte sensor. In some aspects, the processor can be configured to retrieve and store sensor data and the associated unique identifier from the analyte sensor. In some aspects, the processor can be configured to generate a report based on the sensor data. In some aspects, the processor can be configured to upload the report to the EMR account associated with the unique identifier. Advantageously the processor can interface with each of the components of the system (e.g., remote device, analyte sensor), as well as other systems (e.g., remote server, laboratory test facility, order database, etc.), and increase security and integrity of patient data by generating or retrieving the unique identifier associated with the order and the EMR account.

In some aspects, the processor can be configured to activate a professional blinded mode based on the unique identifier such that sensor data is not displayed to the patient. In some aspects, the processor can be configured to activate an unblinded mode based on the unique identifier such that sensor data is displayed to the patient. Advantageously the professional blinded mode can be activated to prevent display of analyte (e.g., glucose) readings to the patient and reduce patient distorted (e.g., biased) analyte reports, due to the patient altering their behavior in response to the displayed analyte readings, for unbiased analyte reports and increased understanding of the patient's analyte (e.g., glycemic) condition by the third party (e.g., PCP, HCP, clinician, etc.).

In some aspects, communication between the analyte measurement system and the remote device can include near-field communication (NFC). In some aspects, communication between the analyte measurement system and the remote device can include Bluetooth Low Energy (BLE). Advantageously NFC between the analyte measurement system (e.g., analyte sensor) and the remote device can increase security and integrity of patient data since no pairing operation is required, communication is limited to a proximate distance (e.g., distance of 4 cm or less), and power to the analyte sensor (e.g., via battery design) can be available after sensor wear (e.g., expiration period of 14 days, etc.) for an indefinite or reasonably long period of time to upload the stored sensor data.

In some aspects, the unique identifier can include a globally unique identifier (GUID), a universally unique identifier (UUID), a unique text string, an analyte sensor serial number, patient information, an identification sticker, a quick-response (QR) code, a two-dimensional (2D) barcode, or a combination thereof. In some aspects, the unique identifier can be coupled to a container configured to return the analyte sensor and/or the remote device to a location of the processor (e.g., a laboratory test facility). In some aspects, the unique identifier can be coupled to the analyte sensor and/or to a container storing the analyte sensor prior to use (e.g., a pre-addressed envelope). Advantageously the unique identifier can increase security and integrity of patient data and can be stored in the analyte sensor when activated to restrict responses to only commands containing the unique identifier (e.g., unique authentication/verification based on a comparison or matching).

In some aspects, the processor can be located at a laboratory test facility. In some aspects, the processor can be in communication with an order interface configured to receive the order from a third party. In some aspects, the processor can be in communication with a result interface configured to provide the report to the third party. Advantageously the processor can streamline requests and delivery of orders (e.g., analyte reports) from third parties (e.g., PCPs, HCPs, clinicians, etc.), reduce third party effort, and update an EMR of the patient (e.g., similar to lab test results).

In some aspects, the remote device can be configured to activate the analyte measurement system based on the unique identifier. In some aspects, the analyte measurement system can be configured to store the unique identifier. Advantageously the unique identifier can increase security and integrity of patient data and the analyte measurement system (e.g., analyte sensor) can restrict responses to only commands containing the unique identifier (e.g., unique authentication/verification based on a comparison or matching).

In some aspects, the remote device can be configured to retrieve the order based on patient information. In some aspects, the remote device can be configured to send patient information to the processor. In some aspects, the remote device can be configured to receive the unique identifier based on a match between the order and the patient information. Advantageously the remote device can provide a means for a user to search and retrieve the order based on patient information (e.g., patient name, patient clinic, patient clinician, patient medical information, etc.) and reduces third party effort.

In some aspects, the remote device can include a second processor configured to monitor a functionality of the analyte measurement system. In some aspects, the second processor can be configured to provide a notification to the patient to indicate measurement of the sensor data is complete. In some aspects, the second processor can execute a second software application. Advantageously the second processor (optional) can provide the patient with the ability to monitor the functionality of their analyte sensor and, for example, can be installed on a patient's smartphone.

In some aspects, the remote device can include a sensor management device configured to be reusable. In some aspects, the remote device can include a sensor management device configured to be disposable. Advantageously the sensor management device (optional) can be included in a kit, along with the analyte sensor and insertion device, so the patient can easily apply and activate the analyte sensor on their own, determine the analyte sensor functionality on their own, use the sensor management device to scan the sensor data on their own, and then either send back the sensor management device to the laboratory test facility (e.g., for reuse) or interface the sensor management device with a computer device to upload the sensor data and dispose of the sensor management device (e.g., disposable single use). Further advantageously the sensor management device reduces in-person patient interactions and time delays (e.g., at clinics, lab test facilities, etc.) since the patient does not need to wait at the clinic or lab facility to verify that the analyte sensor is functional and does not need to return to the clinic or lab facility after sensor wear to upload the sensor data.

In some aspects, the order can be received from a clinic. In some aspects, the order can be received from a PCP. In some aspects, the order can be received from a HCP. In some aspects, the order can be received from a caregiver. Advantageously the system can streamline multiple requests and delivery of analyte reports (e.g., glucose reports) for third parties (e.g., PCPs, HCPs, clinicians, clinics, caregivers, etc.).

In some aspects, the processor can be in communication with a database configured to store the order. In some aspects, the processor can be in communication with a data server configured to store the report. Advantageously the processor can interface with a database (e.g., order database of lab test facility) and/or a data server (e.g., real-world-data server, the Internet) to easily retrieve and store orders and/or reports, for example, for generating confidential (anonymous) big data based on the orders and/or reports.

In some aspects, the analyte can be glucose. In some aspects, the analyte can include multiple analytes (e.g., glucose, ketones, lactic acid, lactose, oxygen, hemoglobin A1C, caffeine, sugars, carbs, etc.). Advantageously the system can streamline requests and delivery of analyte reports (e.g., glucose reports, ketone reports, caffeine reports, etc.), for one or more analytes, to third parties (e.g., PCPs, HCPs, clinicians, etc.) for analyte assessment(s) of the patient.

In some aspects, the processor can execute a software application. In some aspects, the software application can be part of the analyte measurement system. In some aspects, the software application can be part of the remote device. In some aspects, the software application can include a mobile application on the remote device. In some aspects, the software application can be part of the sensor management device. In some aspects, the system can further include a remote server configured to support the software application. Advantageously the software application can all be contained in a patient mobile application (app) or some or part of the software application can be contained in a remote server (e.g., web-server, cloud server, intranet server, etc.) that supports the software application, for example, with processing, communication, and/or reporting functionalities. Further advantageously the software application can include an application programming interface (API) for two or more computer programs to communicate with each other.

In some aspects, a method for analyte assessment of a patient by a third party can include receiving and storing an order for analyte assessment associated with an EMR account of the patient. In some aspects, the method can further include generating or retrieving a unique identifier associated with the order. In some aspects, the method can further include authenticating the unique identifier with a processor in communication with a remote device. In some aspects, the remote device can be configured to receive or retrieve sensor data from an analyte sensor. In some aspects, the method can further include measuring an analyte of the patient with an analyte measurement system in communication with the remote device. In some aspects, the analyte measurement system can include the analyte sensor. In some aspects, the method can further include retrieving and storing sensor data from the remote device based on the order. In some aspects, the method can further include generating a report based on the sensor data. In some aspects, the method can further include uploading the report to the EMR account associated with the unique identifier. Advantageously the method can streamline requests and delivery of analyte reports (e.g., glucose reports) for third parties (e.g., PCPs, HCPs, clinicians, etc.), reduce in-person patient interactions and time delays (e.g., at clinics, lab test facilities, etc.), reduce third party effort, increase security and integrity of patient data, and update an EMR of the patient (e.g., similar to lab test results).

In some aspects, the method can further include activating the analyte measurement system based on the unique identifier. In some aspects, activating can include activating a professional blinded mode based on the unique identifier such that sensor data is not displayed to the patient. Advantageously activating the professional blinded mode with the unique identifier can prevent display of analyte (e.g., glucose) readings to the patient and reduce patient distorted (e.g., biased) analyte reports, due to the patient altering their behavior in response to the displayed analyte readings, for unbiased analyte reports and increased understanding of the patient's analyte (e.g., glycemic) condition by the third party (e.g., PCP, HCP, clinician, etc.).

In some aspects, the method can further include communicating between the analyte measurement system and the remote device. In some aspects, communicating can include NFC. Advantageously NFC between the analyte measurement system (e.g., analyte sensor) and the remote device can increase security and integrity of patient data since no pairing operation is required, communication is limited to a proximate distance (e.g., distance of 4 cm or less), and power to the analyte sensor (e.g., via battery design) can be available after sensor wear (e.g., expiration period of 14 days, etc.) for an indefinite or reasonably long period of time to upload the stored sensor data.

In some aspects, generating the unique identifier can include the processor generating a GUID, a UUID, a unique text string, an identification sticker, a QR code, a 2D barcode, or a combination thereof. In some aspects, the method can further include coupling the unique identifier to a container configured to return the analyte sensor and/or the remote device to a location of the processor (e.g., a laboratory test facility). In some aspects, the method can further include coupling the unique identifier to the analyte sensor and/or to a container storing the analyte sensor prior to use (e.g., a pre-addressed envelope). Advantageously coupling (e.g., affixing) the unique identifier to the sensor analyte, a kit holding the sensor analyte (e.g., prior to use), or a container for returning the sensor analyte after use (e.g., a pre-addressed envelope) can increase security and integrity of patient data (e.g., unique authentication/verification based on a comparison or matching) and reduce in-person patient interactions and time delays.

In some aspects, retrieving the unique identifier can include the processor retrieving an analyte sensor serial number, patient information, an identification sticker, a QR code, a 2D barcode, or a combination thereof. Advantageously the unique identifier can increase security and integrity of patient data and can be retrieved by the processor in a variety of different ways to ensure communications are secure (e.g., unique authentication/verification based on a comparison or matching).

In some aspects, authenticating can include matching patient information with the order. Advantageously the method can provide a means for a user to search and retrieve the order (e.g., matching search) based on patient information (e.g., patient name, patient clinic, patient clinician, patient medical information, etc.) and reduces third party effort.

In some aspects, the method can further include monitoring a functionality of the analyte measurement system. In some aspects, the method can further include notifying the patient when measuring of sensor data is complete. Advantageously the method can provide the patient with the ability to monitor the functionality of their analyte sensor (e.g., separate software application) and, for example, monitoring can be installed on a patient's smartphone (e.g., separate software application).

In some aspects, a glycemic assessment system can include a glucose sensor, a mobile application, and a web application. In some aspects, the glucose sensor can be configured to measure glucose data of a patient. In some aspects, the mobile application can be installed on a mobile device in communication with the glucose sensor. In some aspects, the mobile application can be configured to activate the glucose sensor. In some aspects, the mobile application can be configured to collect glucose data from the glucose sensor. In some aspects, the mobile application can be configured to transmit the glucose data to a remote server. In some aspects, the web application can be in communication with the remote server. In some aspects, the web application can be configured to store the glucose data collected from the glucose sensor. In some aspects, the web application can be configured to generate one or more reports based on the glucose data. In some aspects, the web application can be configured to share the one or more reports with a third party. Advantageously the system can initiate a clinical trial, create an independent storage server (e.g., cloud server) associated with the clinical trial, export sensor data from multiple participants to the independent storage server, streamline requests and delivery of reports (e.g., glucose reports, participant data, clinical trial metrics, etc.), and provide role-based access control for third parties (e.g., PCPs, HCPs, clinicians, research sites, etc.) to review participant data, assess therapy changes, and/or monitor participant health and safety.

In some aspects, the mobile application can be configured to activate a blinded mode such that glucose data is not displayed to the patient. In some aspects, the mobile application can be configured to activate an unblinded mode such that glucose data is displayed to the patient. Advantageously the blinded mode can be activated to prevent display of analyte (e.g., glucose) readings to the patient and reduce patient distorted (e.g., biased) analyte reports, due to the patient altering their behavior in response to the displayed analyte readings, for unbiased analyte reports and increased understanding of the patient's analyte (e.g., glycemic) condition by the third party (e.g., PCP, HCP, clinician, etc.), for example, during a clinical trial.

In some aspects, the system may include a first app for a blinded mode and a second app for an unblinded mode. In some aspects, for example, the first app may include the mobile application configured to operate in a blinded mode such that data (e.g., glucose data, medication dose data, exercise data, meal data, etc.) is not displayed to the patient. In some aspects, for example, the second app may include a mobile application (e.g., similar to the mobile application) configured to operate in an unblinded mode such that data (e.g., glucose data, medication dose data, exercise data, meal data, or a combination thereof, etc.) is displayed to the patient.

In some aspects, the mobile application may operate in a blinded mode such that data (e.g., glucose data, medication dose data, exercise data, meal data, etc.) is not displayed to the patient but is available to a third party for monitoring (e.g., PCP, HCP, clinician, hospital, etc.). In some aspects, for example, the mobile application may provide alerts, reports, titration changes, dose recommendations, etc. to the third party but not display that information to the patient. In some aspects, the data may be blinded for the patient on the mobile application but available to the third party (e.g., PCP, HCP, clinician, hospital, etc.) on the web application. In some aspects, in the blinded mode, if the system determines a need for a change in therapy settings (e.g., insulin dose change), an alert and/or report may be sent to the third party recommending the change (e.g., correction dose) but not to the user. In some aspects, for example, the mobile application may be taken out of the blinded mode into an unblinded mode (e.g., temporarily) after a change in therapy settings (e.g., insulin dose change) so the user may see the change (e.g., confirm change).

In some aspects, the web application may start multiple sensors for multiple patients at the same time. In some aspects, the web application may monitor multiple sensors (e.g., on multiple patients) at the same time and provide corresponding alerts, reports, titration changes, dose recommendations, etc. to the third party (e.g., PCP, HCP, clinician, hospital, etc.). In some aspects, for example, the web application may provide one or more alerts that are color-coded to indicate different types of emergencies (e.g., hypoglycemia, hyperglycemia, cardiac arrest, loss of sensor signal, patient unresponsive, elevated heart rate, elevated temperature, similar to standardized hospital emergency color codes, etc., glucose range color-coded alerts—Red: low glucose reading (e.g., <70 mg/dL), Orange: high glucose reading (e.g., >150 mg/dL), Green: glucose reading in target range (e.g., 110 mg/dL), Yellow: glucose reading between target range and low or high levels, etc.).

In some aspects, the system may include a log book configured to record and store data related to the patient. In some aspects, for example, the log book may operate on the mobile application and receive data from the patient (e.g., medication dose data, exercise data, meal data, etc.) via one or more prompts. For example, the patient may enter “I ate 2 donuts at 10 AM; I injected 2 U of GLP-1 at 11 AM; I worked out 1 PM-3 PM” into the log book for a particular day.

In some aspects, communication between the glucose sensor and the mobile application can include BLE. Advantageously BLE between the glucose sensor and the mobile application can achieve optimized communication (e.g., accurate tracking) and low power consumption, for example, such that power to the glucose sensor can be available after sensor wear (e.g., expiration period of 14 days, etc.) for a reasonably long period of time to upload the stored glucose data.

In some aspects, the web application can be configured to initiate a clinical trial. In some aspects, the web application can be configured to create an independent cloud server on the remote server associated with the clinical trial. Advantageously the independent cloud server created and associated with the clinical trial can store sensor data from multiple participants, as well as participant data and clinical trial metrics, for role-based access control for third parties (e.g., PCPs, HCPs, clinicians, research sites, etc.) to review participant data, assess therapy changes, and/or monitor participant health and safety.

In some aspects, the mobile application can be configured to collect the glucose data over a custom duration. In some aspects, the custom duration is 14 days. Advantageously the custom duration (e.g., 3 days, 7 days, 14 days, 30 days, etc.) can be selected and/or modified based on the clinical trial and review of data during the clinical trial (e.g., can be reduced if participant health is at risk).

In some aspects, the web application can be configured to provide role-based access control for users. In some aspects, the web application can be configured to make therapy changes during the clinical trial. Advantageously the role-based access control for third parties (e.g., PCPs, HCPs, clinicians, research sites, institutions, industry sponsors, CROs, etc.) can provide confidential and secure access to different levels of data associated with the clinical trial based on the specific user, for example, access to data can be configured based on roles and industry sponsor requirements.

In some aspects, the web application can be configured to monitor clinical trial metrics. In some aspects, the clinical trial metrics can include countries conducting the clinical trial, research sites conducting the clinical trial, total number of participants, number of participants per country, number of participants per research site, total usage of participants, or a combination thereof. Advantageously clinical trial metrics can be reviewed and monitored throughout the clinical trial, for example, to monitor clinical trial performance, review usage of product (e.g., analyte sensor), and/or ensure usage is in accordance with business agreements.

Implementations of any of the techniques described above can include a system, a method, a process, a device, and/or an apparatus. The details of one or more implementations are set forth in the accompanying drawings and the description below. Other features will be apparent from the description and drawings, and from the claims.

Further features and exemplary aspects of the aspects, as well as the structure and operation of various aspects, are described in detail below with reference to the accompanying drawings. It is noted that the aspects are not limited to the specific aspects described herein. Such aspects are presented herein for illustrative purposes only. Additional aspects will be apparent to persons skilled in the relevant art(s) based on the teachings contained herein.

### BRIEF DESCRIPTION OF THE DRAWINGS/FIGURES

The accompanying drawings, which are incorporated herein and form part of the specification, illustrate the aspects and, together with the description, further serve to explain the principles of the aspects and to enable a person skilled in the relevant art(s) to make and use the aspects.

FIG. 1 is a schematic illustration of an analyte assessment system with a software application, according to an exemplary aspect.

FIG. 2 is a schematic illustration of an EMR assessment system for requesting, generating, and delivering analyte reports to third parties for analyte assessment of a patient, according to an exemplary aspect.

FIG. 3 is a schematic illustration of a clinical trial assessment system for requesting, generating, and delivering reports associated with a clinical trial to third parties, according to an exemplary aspect.

FIG. 4 illustrates a flow diagram for the EMR assessment system shown in FIG. 2, according to an exemplary aspect.

FIG. 5 illustrates a flow diagram for the clinical trial assessment system shown in FIG. 3, according to an exemplary aspect.

The features and exemplary aspects of the aspects will become more apparent from the detailed description set forth below when taken in conjunction with the drawings, in which like reference characters identify corresponding elements throughout. In the drawings, like reference numbers generally indicate identical, functionally similar, and/or structurally similar elements. Additionally, generally, the left-most digit(s) of a reference number identifies the drawing in which the reference number first appears. Unless otherwise indicated, the drawings provided throughout the disclosure should not be interpreted as to-scale drawings.

### DETAILED DESCRIPTION

This specification discloses one or more aspects that incorporate the features of this present invention. The disclosed aspect(s) merely exemplify the present invention. The scope of the invention is not limited to the disclosed aspect(s). The present invention is defined by the claims appended hereto.

The aspect(s) described, and references in the specification to “one aspect,” “an aspect,” “an example aspect,” “an exemplary aspect,” etc., indicate that the aspect(s) described can include a particular feature, structure, or characteristic, but every aspect may not necessarily include the particular feature, structure, or characteristic. Moreover, such phrases are not necessarily referring to the same aspect. Further, when a particular feature, structure, or characteristic is described in connection with an aspect, it is understood that it is within the knowledge of one skilled in the art to affect such feature, structure, or characteristic in connection with other aspects whether or not explicitly described.

Spatially relative terms, such as “beneath,” “below,” “lower,” “above,” “on,” “upper” and the like, can be used herein for ease of description to describe one element or feature's relationship to another element(s) or feature(s) as illustrated in the figures. The spatially relative terms are intended to encompass different orientations of the device in use or operation in addition to the orientation depicted in the figures. The apparatus can be otherwise oriented (rotated 90 degrees or at other orientations) and the spatially relative descriptors used herein can likewise be interpreted accordingly.

The term “about” or “substantially” or “approximately” as used herein means the value of a given quantity that can vary based on a particular technology. Based on the particular technology, the term “about” or “substantially” or “approximately” can indicate a value of a given quantity that varies within, for example, 0.1-10% of the value (e.g., ±0.1%, ±1%, ±2%, ±5%, or ±10% of the value).

Numerical values, including endpoints of ranges, can be expressed herein as approximations preceded by the term “about,” “substantially,” “approximately,” or the like. In such cases, other aspects include the particular numerical values. Regardless of whether a numerical value is expressed as an approximation, two aspects are included in this disclosure: one expressed as an approximation, and another not expressed as an approximation. It will be further understood that an endpoint of each range is significant both in relation to another endpoint, and independently of another endpoint.

Aspects of the disclosure may be implemented in hardware, firmware, software, or any combination thereof. Aspects of the disclosure may also be implemented as instructions stored on a machine-readable medium (e.g., memory), which may be read and executed by one or more processors. A machine-readable medium may include any mechanism for storing or transmitting information in a form readable by a machine (e.g., a computing device). For example, a machine-readable medium may include read only memory (ROM); random access memory (RAM); magnetic disk storage media; optical storage media; flash memory devices; electrical, optical, acoustic, or other forms of propagated signals (e.g., carrier waves, infrared signals, digital signals, etc.), and other. Further, firmware, software, routines, and/or instructions may be described herein as performing certain actions. However, it should be appreciated that such descriptions are merely for convenience and that such actions in fact result from computing devices, processors, controllers, or other devices executing the firmware, software, routines, instructions, etc.

The term “order for analyte assessment” or “order” as used herein indicates an instruction or authorization from a third party (e.g., PCP, HCP, clinician, etc.) to a provider of medical treatment or services. In some aspects, a third party (e.g., clinician) can simply write an order for a glycemic assessment sensor (e.g., CGM), much like they write an order for lab tests (e.g., blood tests), and when the patient returns for their next visit, the clinician can review the glycemic assessment reports in the patient's EMR (e.g., clinic EMR), just like they do for lab test results. In some aspects, the order can include a prescription (e.g., for medication and/or a medical device), a request (e.g., for glycemic assessment reports), or a referral (e.g., for treatment such as blood tests).

The term “unique identifier” as used herein indicates a unique object or unique class of objects (e.g., physical, digital, conceptual) used to identify or refer to a specific patient and can include a GUID, a UUID, a unique text string, an analyte sensor serial number, patient information, an identification sticker, a QR code, a 2D barcode, or a combination thereof.

The term “third party” or “third parties” as used herein indicates a person or group besides the patient and can include PCPs, HCPs, clinicians, caregivers, research sites, institutions, industry sponsors, CROs, or a combination thereof.

The term “laboratory test facility” or “lab test facility” or “service center” as used herein indicates any laboratory (lab), hospital, clinic, research institution, center, or other testing facility that provides laboratory services to generate reports based on sensor data from a patient.

The term “patient information” as used herein indicates any data pertaining to a patient including, but not limited to, a patient's name, a patient's address, a patient's birthdate, a patient's demographic information, a patient's clinic, a patient's clinician, a patient's medical information (e.g., diagnoses, conditions, medications, medical history, lab results, health insurance, etc.), or a combination thereof.

The term “clinical trial metrics” as used herein indicates metrics, parameters, indicators, or data points of a clinical trial that provide insight or analytics into performance of the clinical trial including, but not limited to, operational metrics, performance metrics, key performance indicators (KPIs), operational performance, countries conducting the clinical trial, research sites conducting the clinical trial, total number of participants, number of participants per country, number of participants per research site, total usage of participants, usage of product (e.g., analyte sensor), usage in accordance with business agreements, number of key opinion leaders (KOLs), number of industry sponsors, number of CROs, or a combination thereof.

### Exemplary Analyte Assessment System

As discussed above, the detection and/or monitoring of analyte levels (e.g., glucose levels) can be vitally important to the health of an individual having diabetes. People with diabetes (PwD) are generally required to monitor their glucose levels to ensure they are maintained within a clinically safe range, and may also use this information to determine if and/or when insulin is needed to reduce glucose levels in their bodies or when additional glucose is needed to raise glucose levels in their bodies. A number of systems allow individuals to monitor their blood glucose, for example, CGM. Some of these systems include electrochemical biosensors, including those that use a glucose sensor adapted to be positioned in vivo, for example, with complete or partial insertion into a subcutaneous or transcutaneous site, within the body for continuous in vivo monitoring of glucose levels from bodily fluids of the site.

In some cases, an analyte sensor (e.g., glucose sensor) is applied and activated for a patient by a third party, for example, a clinician. The patient must then wait a period of time (e.g., an hour) in the clinic after application and activation of the analyte sensor so that the clinician can confirm the analyte sensor (e.g., glucose sensor) is functional and working properly. The clinician activates the new analyte sensor and tests its functionality with a special reader maintained at the clinic. The patient must then return to the clinic after an expiration period of the analyte sensor (e.g., 7 days), with the expired analyte sensor, so that the clinician can scan the expired analyte sensor with the special reader and retrieve the sensor data. The clinician must then upload the sensor data to a separate program or server to generate analyte reports (e.g., glucose reports) for analyte assessment of the patient.

The current procedure for initiating the analyte assessment and viewing the resulting report is arduous and time-consuming for both patients and third parties, including clinicians, PCPs, HCPs, and/or caregivers. The patient must be present (e.g., colocated) with the third party for multiple interactions on separate occasions. Further, patients that can access their sensor data (e.g., unblinded mode) may alter their behavior to correct out-of-range analyte values, which can distort (e.g., bias) the analyte reports and prevent the third party (e.g., PCP, HCP, clinician, etc.) from properly understanding the patient's analyte condition when the patient is not using the analyte sensor.

Aspects of analyte assessment apparatuses, systems, and methods as discussed below can streamline requests and delivery of analyte reports (e.g., glucose reports) for third parties (e.g., PCPs, HCPs, clinicians, etc.), reduce in-person patient interactions and time delays (e.g., at clinics, lab test facilities, etc.), reduce third party effort, increase security and integrity of patient data, update an EMR of the patient (e.g., similar to lab test results), and operate in a blinded mode for unbiased patient analyte assessment. Further, aspects of analyte assessment apparatuses, systems, and methods as discussed below can initiate a clinical trial, create an independent storage server (e.g., cloud server) associated with the clinical trial, export sensor data from multiple participants to the independent storage server, streamline requests and delivery of reports (e.g., glucose reports, participant data, clinical trial metrics, etc.), and provide role-based access control for third parties (e.g., PCPs, HCPs, clinicians, research sites, institutions, industry sponsors, CROs, etc.) to review participant data, assess therapy changes, monitor participant health and safety, and/or modify parameters of the clinical trial.

In some aspects, an analyte assessment system can include an analyte sensor, a remote device, and a software application. The remote device can be configured to retrieve sensor data of a patient from the analyte sensor. The software application can be configured to receive an order for analyte assessment associated with an EMR of the patient, generate or retrieve a unique identifier associated with the order, authenticate the unique identifier with the remote device (e.g., verification based on a comparison or matching of the unique identifier with patient information, analyte sensor serial number, QR sticker, or a combination thereof), activate the analyte sensor based on the unique identifier, retrieve sensor data from the remote device, generate a report, and upload the report to the EMR of the patient. Advantageously the analyte assessment system can streamline requests and delivery of analyte reports for third parties, reduce in-person patient interactions and time delays, reduce third party effort, increase security and integrity of patient data, update an EMR of the patient, and operate in a blinded mode for unbiased patient analyte assessment.

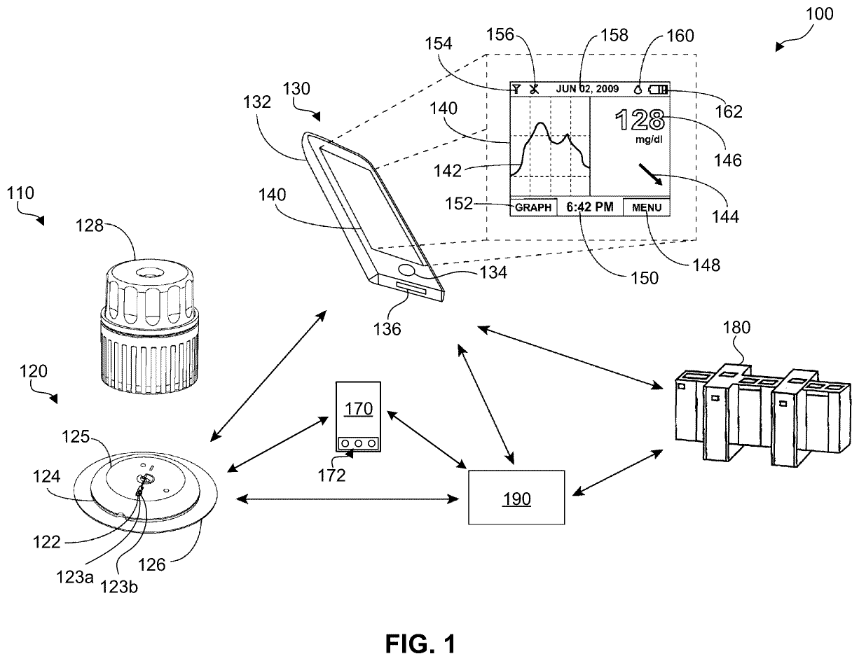

FIG. 1 illustrates analyte assessment system 100 with software application 190, according to exemplary aspects. Analyte assessment system 100 can be configured to streamline requests and delivery of analyte reports (e.g., glucose reports) for third parties (e.g., PCPs, HCPs, clinicians, etc.) and reduce in-person patient interactions and time delays (e.g., at clinics, lab test facilities, etc.). Analyte assessment system 100 can be further configured to reduce third party effort and update an EMR of the patient (e.g., similar to lab test results). Analyte assessment system 100 can be further configured to increase security and integrity of patient data and operate in a blinded mode for unbiased patient analyte assessment. Although analyte assessment system 100 is shown in FIG. 1 as a stand-alone apparatus and/or system, aspects of this disclosure can be used with other apparatuses, systems, and/or methods, for example, EMR assessment system 200, clinical trial assessment system 300, flow diagram 400, and/or flow diagram 500.

In some aspects, components of analyte assessment system 100 can include a sensor (e.g., analyte sensor 122), sensor electronics (e.g., on body electronics 124) that can store sensor data (e.g., 3 days of data, 7 days of data, 14 days of data, 30 days of data, etc.), an applicator (e.g., insertion device 128), an electronic device (e.g., remote device 130 and/or sensor management device (SMD) 170) for activating, monitoring, and uploading data from the sensor, and a glucose data reporting system (e.g., software application 190). In some aspects, analyte assessment system 100 can provide a method and system for third parties (e.g., PCPs, HCPs, clinicians, etc.) to access and review a patient's glucose data that fits into the current clinical workflow. For example, a third party (e.g., clinician) can simply write an order for a glycemic assessment sensor, much like they write an order for lab tests (e.g., blood tests), and when the patient returns for their next visit, the clinician can review the glycemic assessment reports in the patient's EMR (e.g., clinic EMR), just like they do for lab test results. In some aspects, the third party (e.g., clinician) can view these generated reports in the EMR at any time that is convenient, as well, because the patient does not need to be present with the clinician.

In some aspects, after the third party (e.g., clinician) writes up an order for glycemic assessment, the patient eventually goes to a lab test facility, where they would normally go to get their lab tests, and the sensor would be applied and activated there. In some aspects, alternatively, the patient can pick up a sensor kit and apply the sensor at home at a time of their convenience. For example, the “kit” can contain the sensor (e.g., analyte sensor 122), the applicator (e.g., insertion device 128), user instructions, a sensor management device (e.g., SMD 170), a self-addressed envelope, and an identification (ID) card. In some aspects, analyte assessment system 100 can include the sensor “kit” and designed such that a patient can easily apply and activate the sensor on their own after they have received it. In some aspects, analyte assessment system 100 allows a patient to determine the sensor functionality on their own. In some aspects, at a lab test facility or service center, a sticker can be printed out with the patient information and clinic identifying information, which can be applied to the ID card and/or directly to the sensor management device (e.g., SMD 170). For example, this information (e.g., ID card, SMD 170) can be mailed back to the service center for data upload, and subsequent result upload to the clinical EMR for the specific patient account.

As shown in FIG. 1, analyte assessment system 100 can include analyte measurement system 110, remote device 130, sensor management device (SMD) 170, remote server 180, and/or software application 190. Analyte measurement system 110 can be configured to measure (e.g., via on body unit 120) an analyte (e.g., glucose) of a patient. Analyte measurement system 110 can be further configured to display and/or notify (e.g., via remote device 130) a patient of measured levels of an analyte (e.g., glucose). As shown in FIG. 1, analyte measurement system 110 can include on body unit (OBU) 120 and insertion device 128.

OBU 120 can be configured to measure and communicate sensor data of an analyte (e.g., glucose) of a patient. OBU 120 can be further configured to communicate data (e.g., sensor data) from analyte sensor 122 to one or more components of analyte assessment system 100 (e.g., remote device 130, SMD 170, remote server 180, software application 190, etc.). As shown in FIG. 1, OBU 120 can include analyte sensor 122, on body electronics 124, on body housing 125, and/or adhesive layer 126.

Analyte sensor 122 can be configured to measure an analyte (e.g., glucose) of a patient. Analyte sensor 122 can be configured to measure one or more analytes (e.g., glucose, ketones, lactic acid, etc.) of a patient. Analyte sensor 122 can be further configured to continuously measure (e.g., in vivo) in real-time a concentration of one or more analytes (e.g., first analyte 123a, second analyte 123b, etc.) of a patient. As shown in FIG. 1, analyte sensor 122 can detect first analyte 123a (e.g., glucose) and second analyte 123b (e.g., ketone). In some aspects, a portion of analyte sensor 122 (e.g., distal portion) can be positioned in vivo through a skin surface of a patient (e.g., transcutaneously) and in fluid contact with bodily fluids (e.g., blood, interstitial fluid, etc.) of the patient. In some aspects, analyte sensor 122 can be insertable into a body of a patient (e.g., vein, artery, skin, etc.) containing an analyte. In some aspects, analyte sensor 122 can include CGM to continuously and automatically track glucose levels.

In some aspects, for example, as shown in FIG. 1, analyte sensor 122 can include a single (dual) analyte sensor to measure first and second analytes 123a, 123b (e.g., simultaneously). In some aspects, analyte sensor 122 can include two separate analyte sensors to measure first and second analytes 123a, 123b (e.g., a CGM sensor and a separate continuous ketone monitoring sensor). In some aspects, first analyte 123a can be glucose and second analyte 123b can be ketones. In some aspects, analyte sensor 122 can measure and retrieve glucose and ketone levels in real-time (e.g., about 1-60 seconds) for continuous analyte (glucose-ketone) monitoring. In some aspects, analyte sensor 122 can measure and retrieve glucose and ketone levels in near real-time (e.g., about 1-15 minutes) for discrete analyte (glucose-ketone) monitoring.

In some aspects, analyte sensor 122 can automatically and/or continuously monitor one or more analyte levels (e.g., first analyte 123a, second analyte 123b, etc.) in vivo, for example, glucose and ketones of a patient, over a predetermined time interval (e.g., sensor lifetime) or given sensing period (e.g., 1 day, 3 days, 7 days, 14 days, 30 days, etc.). In some aspects, analyte sensor 122 can be coupled (e.g., electronically) to on body electronics 124 to process information obtained from analyte sensor 122 (e.g., sensor data). In some aspects, analyte sensor 122 can be in communication (e.g., wired, wirelessly) with on body electronics 124.

In some aspects, analyte sensor 122 can measure glucose. In some aspects, analyte sensor 122 can measure one or more analytes. For example, analyte sensor 122 can measure one or more metabolic analytes (e.g., glucose, ketones, lactate, lactic acid, oxygen, hemoglobin A1C, lactone, lactose, galactose, vitamin C, glucoronate, glycogen, mannose, phosphate, bisphosphate, fructose, glyceraldehyde, glycerol, triglycerides, sorbitol, phosphoglucono, phosphogluconate, xylulose, ribose, bile, cysteine, serine, homoserine, pyruvate, phenylpyruvate, glutamate, glycine, taurine, threonine, methionine, ethanol, acetone, acetate, oxaloacetate, alanine, phenylalanine, aspartate, asparagine, alcohol, cholesterol, vitamin D, progesterone, testosterone, estrogen, squalene, insulin, hydroxybutyrate, leucine, isoleucine, malonyl, malonate, glucagon, epinephrine, norepinephrine, palmitate, lysine, eicosanoids, melanin, dopamine, tyrosine, tryptophan, niacin, melatonin, serotonin, citrate, isocitrate, valine, porphyrins, histidine, urocanate, histamine, glutamine, proline, creatine, putrescine, spermidine, spermine, arginine, ornithine, citrulline, fumarate, succinate, argininosuccinate, succinyl, ketoglutarate, aconitate, glyoxylate, caffeine, sugars, carbs, etc.). In some aspects, analyte sensor 122 can measure one or more analytes simultaneously with one or more corresponding electrochemical biosensors for each different analyte measured.

On body electronics 124 can be configured to process signals from analyte sensor 122. On body electronics 124 can be further configured to communicate data (e.g., sensor data) from analyte sensor 122 to one or more external devices (e.g., remote device 130, SMD 170, remote server 180, software application 190, etc.). On body electronics 124 can be further configured to wirelessly communicate (e.g., NFC, WiFi, Bluetooth, BLE, Internet, etc.) analyte related sensor data (e.g., first analyte 123a and/or second analyte 123b). As shown in FIG. 1, on body electronics 124 can be operatively (e.g., electrically) coupled to analyte sensor 122 and wirelessly coupled to remote device 130, SMD 170, and/or software application 190.

In some aspects, on body electronics 124 can include a printed circuit board (PCB) for connection to various components (e.g., analyte sensor 122, processor, ASIC, wireless transceiver, wireless transmitter, controller, memory, etc.). In some aspects, on body electronics 124 can store (e.g., via memory) historical analyte related data (e.g., first analyte 123a and/or second analyte 123b). In some aspects, on body electronics 124 can be configured to store some or all of analyte related data (e.g., sensor data) from analyte sensor 122 in a memory, for example, during a sensing period (e.g., 1 day, 3 days, 7 days, 14 days, 30 days, etc.). In some aspects, on body electronics 124 can include one or more processors and/or control logic configured to determine (e.g., via software programs and/or algorithms) current analyte levels (e.g., first analyte level, second analyte level, etc.), rates of change of analyte levels (e.g., first analyte ROC, second analyte ROC, etc.), rates of acceleration of analyte levels (e.g., rate of first analyte ROC, rate of second analyte ROC, etc.), and/or analyte trend information (e.g., trend display 144), and/or analyte fluctuation levels (e.g., standard deviation, etc.).

In some aspects, on body electronics 124 can be configured to send (broadcast) analyte related data (e.g., sensor data) to one or more external devices (e.g., remote device 130, SMD 170, remote server 180, software application 190, etc.), for example, after receiving a command or request from the external device. In some aspects, on body electronics 124 can be configured to send (broadcast) real-time data associated with monitored analyte levels (e.g., first analyte 123a, second analyte 123b, etc.) from analyte sensor 122 to one or more external devices (e.g., remote device 130, SMD 170, remote server 180, software application 190, etc.), for example, when the external device is within a communication range (e.g., NFC range) of the data broadcast from OBU 120.

In some aspects, on body electronics 124 can be configured to wirelessly transmit stored analyte related sensor data (e.g., first analyte 123a, second analyte 123b, etc.) during a monitoring time period (e.g., sensor wear) to one or more external devices (e.g., remote device 130, SMD 170, remote server 180, software application 190, etc.). In some aspects, analyte related data (e.g., sensor data) sent from on body electronics 124 can be stored in one or more memory units (e.g., permanently, temporarily), for example, memory units on one or more external devices (e.g., remote device 130, SMD 170, remote server 180, software application 190, etc.). In some aspects, remote device 130 can be configured as a data conduit to pass data received from on body electronics 124 (e.g., sensor data) to one or more external devices (e.g., remote server 180, software application 190, etc.). In some aspects, SMD 170 can be configured as a data conduit to pass data received from on body electronics 124 (e.g., sensor data) to one or more external devices (e.g., remote server 180, software application 190, etc.).

In some aspects, on body electronics 124 can be designed to store the sensor data (e.g., glucose data) from analyte sensor 122 collected over the course of the sensor wear period (e.g., 3 days, 7 days, 14 days, 30 days, etc.), for example, over 14 days. In some aspects, on body electronics 124 can be designed to only communicate with remote device 130 via NFC. In some aspects, on body electronics 124 can be designed to only communicate with SMD 170 via NFC.

On body housing 125 can be configured to provide an interior compartment for a portion of analyte sensor 122 (e.g., proximal portion) and on body electronics 124. As shown in FIG. 1, on body housing 125 can include analyte sensor 122 and on body electronic 124 and be coupled to adhesive layer 126. In some aspects, on body housing 125 can include a sealed housing (e.g., hermetically sealed biocompatible housing). Adhesive layer 126 can be configured to attach OBU 120 to a skin surface of a patient. As shown in FIG. 1, adhesive layer 126 can be coupled to on body housing 125 to securely position a portion of analyte sensor 122 (e.g., distal portion) to a skin surface. In some aspects, adhesive layer 126 can provide a terminal seal of insertion device 128.

Insertion device 128 can be configured to position a portion of analyte sensor 122 (e.g., distal portion) through a skin surface of a patient (e.g., in vivo) and in fluid contact with bodily fluids (e.g., blood, interstitial fluid) of the patient. Insertion device 128 can be further configured to adhere OBU 120 onto the skin surface of a patient. As shown in FIG. 1, insertion device 128 can be configured to hold OBU 120 and, when operated, position a portion of analyte sensor 122 in vivo through a skin surface of a patient and in fluid contact with bodily fluids (e.g., blood, interstitial fluid), and secure OBU 120 to the skin surface. In some aspects, OBU 120 can be sealed within insertion device 128 prior to use.

Remote device 130 can be configured to receive or retrieve sensor data from analyte sensor 122. Remote device 130 can be further configured to activate analyte sensor 122. Remote device 130 can be further configured to transmit sensor data from analyte sensor 122 to software application 190 (e.g., after authentication or transfer of unique identifier to remote device 130). Remote device 130 can be configured to operate in a blinded mode for unbiased patient analyte assessment. As shown in FIG. 1, remote device 130 can be operatively (e.g., wirelessly, NFC, BLE, etc.) coupled to OBU 120, remote server 180, and/or software application 190. In some aspects, remote device 130 can include a handheld computer (e.g., smartphone, cell phone, mobile phone, PDA, smart watch, etc.), personal computer, laptop computer, or any other portable communication device.

In some aspects, remote device 130 can be configured to monitor a functionality of analyte measurement system 110 (e.g., functionality of analyte sensor 122). In some aspects, remote device 130 can be configured to provide a notification to the patient (e.g., indicating measurement of sensor data is complete, indicating analyte sensor 122 expiration time, indicating sensor data error, indicating analyte sensor 122 error, etc.). In some aspects, remote device 130 can be omitted from analyte assessment system 100. For example, SMD 170 can be used in place of remote device 130.

In some aspects, communication between analyte measurement system 110 (e.g., analyte sensor 122) and remote device 130 can be NFC. In some aspects, remote device 130 can be configured to activate analyte measurement system 110 (e.g., analyte sensor 122) based on a unique identifier. In some aspects, analyte measurement system 110 (e.g., analyte sensor 122) can be configured to store the unique identifier. In some aspects, the unique identifier can include a GUID, a UUID, a unique text string, an analyte sensor serial number, patient information, an identification sticker, a QR code, a 2D barcode, or a combination thereof. In some aspects, remote device 130 can be configured to activate a professional blinded mode based on a unique identifier (e.g., GUID, UUID, etc.) such that sensor data is not displayed to the patient.

In some aspects, remote device 130 can be configured to retrieve an order for patient assessment (e.g., from a third party) based on patient information. In some aspects, remote device 130 can be configured to send patient information to software application 190. In some aspects, remote device 130 can be configured to receive a unique identifier (e.g., GUID, UUID, etc.) based on a match between the order and the patient information.

In some aspects, remote device 130 can be configured to monitor a functionality of analyte measurement system 110 (e.g., analyte sensor 122). In some aspects, remote device 130 can include a mobile application (app) and a processor configured to execute instruction of the mobile app. In some aspects, the mobile app can be configured to provide a notification (e.g., alert, timer, text, etc.) to indicate measurement of sensor data from analyte sensor 122 is complete, for example, in a professional blinded mode.

In some aspects, a default home screen of remote device 130 can include analyte data (e.g., analyte level, analyte rate of change, analyte trend, etc.). In some aspects, the default home screen of remote device 130 can include glucose data (e.g., glucose level, glucose rate of change, glucose trend, etc.). In some aspects, the default home screen of remote device 130 can monitor a functionality of analyte measurement system 110 (e.g., analyte sensor 122). As shown in FIG. 1, remote device 130 can include housing 132, input component 134, data communication port 136, and/or display 140.

Input component 134 can be configured to control operation of remote device 130. Input component 134 can be further configured to input data and/or commands to remote device 130. As shown in FIG. 1, input component 134 can interact with remote device 130 to control operation of remote device 130. In some aspects, input component 134 can include a button, an actuator, a switch, a job wheel, a touch screen, a microphone, a camera, a combination thereof, or a similar input element. For example, input component 134 can be a touch screen or touch sensitive element of display 140.

Data communication port 136 can be configured to communicate data with one or more external devices (e.g., OBU 120, SMD 170, remote server 180, software application 190, blood glucose reader, etc.). As shown in FIG. 1, data communication port 136 can be operatively coupled to housing 132 of remote device 130. In some aspects, data communication port 136 can include wireless data communication (e.g., NFC, BLE, WiFi, Bluetooth, cloud computing, Internet, etc.). In some aspects, data communication port 136 can include a wireless transceiver, wireless transmitter, and/or wireless receiver. In some aspects, data communication port 136 can include wired data communication (e.g., USB port, mini-USB port, serial port, Ethernet port, Internet port, etc.).

In some aspects, data communication port 136 can be configured to receive data from an in vitro test strip (e.g. having a fluid sample thereon) based on in vitro measurements (e.g., blood glucose measurement, etc.), for example, from a blood glucose reader. In some aspects, data communication port 136 can be configured to receive data from an in vitro glucose test strip based on in vitro blood glucose measurements via a blood glucose reader. In some aspects, data communication port 136 can be configured to receive data from an in vitro test strip based on in vitro fluid measurements for a variety of analytes (e.g., glucose, ketones, lactate, lactic acid, oxygen, hemoglobin A1C, etc.), via a fluid analyte reader.

Display 140 can be configured to display a variety of information—some or all of which can be displayed at the same time or at different times. Display 140 can be further configured to output alarms, notifications (e.g., warnings, recommendations, guidance, etc.), prompts, first analyte 123a (e.g., glucose) levels, second analyte 123b (e.g., ketone) levels, or a combination thereof, which can be visual, audio, tactile, or a combination thereof. As shown in FIG. 1, display 140 can include, but is not limited to, graphical display 142, trend display 144, numerical display 146, menu input 148, time display 150, graph input 152, connectivity display 154, alarm display 156, date display 158, sensor calibration display 160, and/or battery display 162.

In some aspects, a default home screen for display 140 can display glucose data, for example, the glucose level (e.g., numerical display 146), rate of change of glucose level (e.g., trend display 144), and a glucose plot (e.g., graphical display 142). In some aspects, a default home screen for display 140 can omit all analyte data. For example, in a blinded mode (e.g., professional blinded mode), no analyte data (e.g., no glucose data) is displayed on display 140 of remote device 130.

Graphical display 142 can be configured to provide a graphical plot (e.g., time series plot) of first analyte 123a (e.g., glucose) and/or second analyte 123b (e.g., ketone) from analyte sensor 122. As shown in FIG. 1, graphical display 142 can include a plot of first analyte 123a (e.g., glucose) over time. In some aspects, graphical display 142 can include a plot of second analyte 123b (e.g., ketones) over time. In some aspects, graphical display 142 can include one or more plots of corresponding one or more analytes. For example, graphical display 142 can include a glucose time series plot (e.g., based on first analyte 123a) and a ketone time series plot (e.g., based on second analyte 123b) colocated on display 140. In some aspects, graphical display 142 can include important markers, for example, meals, exercise, sleep, heart rate, blood pressure, etc.

Trend display 144 can be configured to indicate a rate of change (ROC) of first analyte 123a (e.g., glucose) and/or second analyte 123b (e.g., ketone). As shown in FIG. 1, trend display 144 can include an arrow (trend) indicating a ROC of first analyte 123a (e.g., glucose). In some aspects, trend display 144 can indicate a magnitude and a direction of any ongoing trend, for example, a ROC of first analyte 123a (e.g., glucose). In some aspects, trend display 144 can include a ROC of first analyte 123a (e.g., glucose). In some aspects, trend display 144 can include a ROC of second analyte 123b (e.g., ketone). In some aspects, trend display 144 can include one or more arrows (trends) of corresponding one or more analytes.

Numerical display 146 can be configured to provide monitored levels of first analyte 123a (e.g., glucose) and/or second analyte 123b (e.g., ketone). As shown in FIG. 1, numerical display 146 can indicate a current value of an analyte, for example, first analyte 123a (e.g., glucose). In some aspects, numerical display 146 can include a numerical level of first analyte 123a (e.g., glucose), for example, in units of mg/dL. In some aspects, numerical display 146 can include a numerical level of second analyte 123b (e.g., ketone), for example, in units of mmol/L.

Menu input 148 can be configured to control operation of remote device 130. Menu input 148 can be further configured to provide access to additional menus of display 140 (e.g., changing display settings, etc.). As shown in FIG. 1, menu input 148 can be located on display 140. In some aspects, menu input 148 can be a touch screen button or touch sensitive element of display 140. In some aspects, menu input 148 can include a touch screen, gesture recognition, command recognition (e.g., audio, visual), or other suitable input element. In some aspects, menu input 148 can be configured to change display configurations of display 140, for example, changing default home screen configurations.

Time display 150 can be configured to provide time of day information. Connectivity display 154 can be configured to indicate wireless communication connections with other devices (e.g., OBU 120, SMD 170, remote server 180, software application 190, etc.). Date display 158 can be configured to provide date information. Battery display 162 can be configured to indicate (e.g., graphically) a condition of the battery (e.g., rechargeable, disposable) of remote device 130. As shown in FIG. 1, time display 150, connectivity display 154, date display 158, and battery display 162 can be located along a perimeter panel of display 140.

Graph input 152 can be configured to control operation of graphical display 142. In some aspects, graph input 152 can be further configured to provide access to additional menus of graphical display 142 (e.g., changing graphical display settings, etc.). As shown in FIG. 1, graph input 152 can be a touch screen button or touch sensitive element of display 140. In some aspects, graph input 152 can include a touch screen, gesture recognition, command recognition (e.g., audio, visual), or other suitable input element. In some aspects, graph input 152 can be configured to change display configurations of graphical display 142, for example, changing a time scale (X-axis) of graphical display 142.

Alarm display 156 can be configured to indicate a status of an alarm state. As shown in FIG. 1, alarm display 156 can be located above graphical display 142 and indicate when particular alarms have been triggered (e.g., via software application 190). In some aspects, alarm display 156 can indicate one or more particular alarms. In some aspects, graphical display 142 can also display alarm icons in conjunction with alarm display 156. In some aspects, particular alarms and/or alarm icons can be changed (e.g., customized) by a user or third party (e.g., PCP, HCP, clinician, etc.).

Sensor calibration display 160 can be configured to indicate when calibration of analyte sensor 122 is necessary. As shown in FIG. 1, sensor calibration display 160 can be located on an upper panel of display 140. In some aspects, sensor calibration display 160 can provide periodic, routine, and/or predetermined calibration events based on a status of analyte sensor 122. In some aspects, sensor calibration display 160 can notify a user when calibration or replacement of analyte sensor 122 is needed, for example, display 140 (e.g., graphical display 142, alarm display 156) can also display calibration alarm icons in conjunction with sensor calibration display 160. In some aspects, sensor calibration can be omitted (e.g., calibration not needed).

SMD 170 can be configured to reduce in-person patient interactions and time delays (e.g., at clinics, lab test facilities, etc.). SMD 170 can be further configured to receive or retrieve sensor data from analyte sensor 122. SMD 170 can be further configured to activate analyte sensor 122. SMD 170 can be further configured to transmit sensor data from analyte sensor 122 to software application 190 (e.g., after authentication or transfer of unique identifier to SMD 170). SMD 170 can be further configured to operate in a blinded mode for unbiased patient analyte assessment. As shown in FIG. 1, SMD 170 can be operatively (e.g., wirelessly, NFC, BLE, etc.) coupled to OBU 120 and/or software application 190. In some aspects, SMD 170 can include a handheld computer (e.g., smartphone, cell phone, mobile phone, PDA, smart watch, reader, etc.), personal computer, laptop computer, or any other portable communication device. In some aspects, SMD 170 can be used in place of remote device 130.

In some aspects, SMD 170 can be releasably coupled to analyte measurement system 110 (e.g., detachable). For example, SMD 170 can be a releasable component of OBU 120. In some aspects, SMD 170 can be configured to provide a notification to the patient (e.g., indicating measurement of sensor data is complete, indicating analyte sensor 122 expiration time, indicating sensor data error, indicating analyte sensor 122 error, etc.). In some aspects, SMD 170 can be reusable. In some aspects, SMD 170 can be disposable, for example, SMD 170 can be a single use device.

In some aspects, SMD 170 can be included in a kit, along with analyte measurement system 110 (e.g., OBU 120 and insertion device 128), so a patient can easily apply and activate analyte sensor 122 on their own, determine analyte sensor 122 functionality on their own, and use SMD 170 to scan the sensor data on their own. In some aspects, after transferring sensor data from analyte sensor 122 to SMD 170, the patient can send back SMD 170 to a third party (e.g., lab test facility, sensor manufacturer, etc.) for reuse, for example, in another analyte sensor (e.g., disinfected, recharged, reset, etc.). In some aspects, after transferring sensor data from analyte sensor 122 to SMD 170, the patient can interface SMD 170 with a computer device (e.g., via software application 190) to upload the sensor data and then dispose of SMD 170 (e.g., disposable single use).

In some aspects, communication between analyte measurement system 110 (e.g., analyte sensor 122) and SMD 170 can be NFC. In some aspects, SMD 170 can be configured to activate analyte measurement system 110 (e.g., analyte sensor 122) based on a unique identifier. In some aspects, analyte measurement system 110 (e.g., analyte sensor 122) can be configured to store the unique identifier. In some aspects, SMD 170 can be configured to activate a professional blinded mode based on a unique identifier (e.g., GUID, UUID, etc.) such that sensor data is not displayed to the patient.

In some aspects, the unique identifier can include a GUID, a UUID, a unique text string, an analyte sensor serial number, patient information, an identification sticker, a QR code, a 2D barcode, or a combination thereof. In some aspects, SMD 170 can be configured to activate a professional blinded mode based on a unique identifier (e.g., GUID, UUID, etc.) such that sensor data is not displayed to the patient. In some aspects, the unique identifier can be coupled to a container (e.g., self-addressed envelope) configured to return analyte sensor 122 and/or SMD 170 to a location of software application 190 (e.g., a lab test facility). In some aspects, the unique identifier can be coupled to analyte sensor 122 and/or to a container storing analyte sensor 122 prior to use.

In some aspects, SMD 170 can be optional. For example, analyte assessment system 100 can omit SMD 170 and utilize remote device 130 instead. In some aspects, remote device 130 can be optional. For example, analyte assessment system 100 can omit remote device 130 and utilize SMD 170 instead.

In some aspects, once the patient has completed the sensor wear (e.g., 3 days, 7 days, 14 days, 30 days, etc.), the patient can use SMD 170 to scan the sensor data (e.g., glucose data), and mail SMD 170 to a service center where the sensor data can be uploaded from it and used to generate glycemic data, reports, and metrics. For example, the sensor data from SMD 170 can be uploaded into a clinic's EMR the same way lab results are uploaded (e.g., blood results), and the third party (e.g., clinician) can view the results in the same way that lab test results are viewed today.

In some aspects, the lab test facility can utilize a current procedural terminology (CPT) code (e.g., categorized codes that offer PCPs, HCPs, clinicians, etc. a uniform language for coding medical services and procedures, e.g., 80047-89398: Pathology and Laboratory Procedures, 90281-99607: Medicine Services and Procedures, etc.) for billing professional sensor application and report generation. In some aspects, the upload and report generation service can be part of a lab test facility or a separate service. In some aspects, the upload of the results to the requesting third party (e.g., a clinic) can be in the same way that lab test results are currently uploaded to a clinic's EMR.

In some aspects, SMD 170 can be an inexpensive electronic device. In some aspects, SMD 170 can include one or more light emitting diodes (LEDs) to communicate with the user. For example, as shown in FIG. 1, SMD 170 can include LEDs 172. In some aspects, SMD 170 can activate analyte sensor 122, interrogate analyte sensor 122 to determine its functionality and state, and upload sensor data from analyte sensor 122 (e.g., via on body electronics 124). In some aspects, SMD 170 can also interface electronically with a computing device (e.g., a computer, tablet, smartphone, reader etc.), for example, through an inexpensive data communication means (e.g., USB port, mini-USB port, serial port, Ethernet port, Internet port, etc.), in order for the computing device to retrieve the sensor data (e.g., glucose data).

In some aspects, a lab test facility can generate one or more unique stickers to place on the test specimens (e.g., on analyte sensor 122 and/or SMD 170), where these stickers contain identification information of the patient, the clinic, and/or the third party (e.g., HCP) that ordered the test assessment. In some aspects, the identification information can be encoded, for example, as in a QR code. For example, the encoded identification information (e.g., QR code) can increase security and integrity of patient data, and ensure that lab results are not erroneously applied to the wrong patient or sent to the wrong third party (e.g., HCP). In some aspects, one or more unique stickers (e.g., QR code stickers) can be generated and placed on analyte sensor 122 and/or SMD 170.

In some aspects, a computer software program (e.g., software application 190) can be used to program the identification information directly into SMD 170. In some aspects, this identification information can be passed on and stored in OBU 120 (e.g., in on body electronics 124) and associated with the sensor data (e.g., glucose data). For example, when the glucose data are uploaded to a lab test facility (e.g., via software application 190), the uploaded data can include the identification information. In some aspects, the identification information can be contained in SMD 170 and associated with the sensor data (e.g., glucose data) once it is retrieved from analyte sensor 122. For example, in this case, analyte sensor 122 can be designed so that it can be activated by any sensor management device (e.g., SMD 170), but subsequent communications can only be performed with the sensor management device that had activated analyte sensor 122 (e.g., SMD 170).

In some aspects, SMD 170 can be designed to only activate a single sensor (e.g., analyte sensor 122), for example, the sensor that SMD 170 is packaged with (e.g., in a sensor “kit”), until SMD 170 is reset for reuse with a different sensor. In some aspects, after SMD 170 has been used to upload sensor data (e.g., glucose data), SMD 170 can be sent back to a lab test facility and/or a manufacturer, disinfected, battery recharged, and software reset, so that SMD 170 can be used with another sensor (e.g., reusable). In some aspects, alternatively, SMD 170 can be designed as a disposable single use device.

In some aspects, SMD 170 can include visual indicators to communicate with a user. For example, as shown in FIG. 1, SMD 170 can include LEDs 172 (e.g., low-cost dynamic colored LEDs). In some aspects, SMD 170 can include an input component (e.g., a button, an actuator, a switch, a job wheel, a touch screen, a microphone, a camera, a combination thereof, or an input element similar to input component 134 of remote device 130) to help initiate activation and prevent inadvertent activation. In some aspects, SMD 170 can utilize visual indicators (e.g., LEDs 172) to communicate activation of analyte sensor 122. For example, one illuminated LED of LEDs 172 (e.g., green color) could indicate successful activation. In some aspects, LEDs 172 (e.g., dynamic colors) can be configured to indicate different function states of SMD 170. For example, one color can indicate a warmup state (e.g., yellow color), a second color can indicate normal operation (e.g., orange color), and a third color (e.g., blue color) can indicate that the normal operation is complete and analyte sensor 122 is ready for data upload.

In some aspects, SMD 170 can avoid the patient waiting at the lab test facility or service center to check if analyte sensor 122 is functional. In some aspects, SMD 170 can avoid the patient returning to the lab test facility or service center after sensor wear (e.g., 14 days) to upload the sensor data. In some aspects, when the patient is done with the sensor wear (e.g., after 14 days), the patient can scan analyte sensor 122 with SMD 170 (e.g., via NFC), mail SMD 170 to the lab test facility or service center, and dispose of the used sensor (e.g., OBU 120).

In some aspects, when SMD 170 is received at the service center, SMD 170 can be connected to a computing device (e.g., a computer, tablet, smartphone, reader, etc.) and the sensor data can be uploaded. In some aspects, a software program (e.g., software application 190) can be used to generate reports from the sensor data (e.g., glucose reports). In some aspects, a software program (e.g., software application 190) can also display the identification information.

In some aspects, the software program (e.g., software application 190) can also provide an interface where an operator can indicate what medications the patient is taking (e.g., diabetes medications), for example, if the operator has access to the patient medical record through the EMR. In some aspects, alternatively, the medication information can be entered by a third party (e.g., PCP, HCP, clinician, etc.) in the order and this could be entered into SMD 170 prior to activating analyte sensor 122, or entered into a report generation software program (e.g., software application 190). In some aspects, the medication information can be used with the sensor data to generate an analyte report that provides therapy considerations for the clinician specific to the medication information entered. For example, diabetes medication information can be used with the glucose data to generate a glucose report that provides therapy considerations for the clinician specific to the diabetes medication information entered.

In some aspects, therapy considerations can include (a) patients using long acting insulin, (b) patients that are candidates to start long acting insulin, and (c) patients that are candidates to start multiple daily injection (MDI) insulin therapy. For example, for patients using long acting insulin (e.g., no meal-time insulin), the report can provide a hyperlink to information about a sensor-based basal titration kit. For example, for patients that are candidates to start long acting insulin, the report can provide a hyperlink to a sensor-based basal start and titration kit. For example, for patients that are candidates to start MDI therapy, the report can provide a hyperlink to a sensor-based prandial insulin start and titration kit.

In some aspects, analyte assessment system 100 can include a mobile application (app) option. In some aspects, a mobile app (e.g., installed on remote device 130) can be designed to serve the purpose of SMD 170. For example, as shown in FIG. 1, remote device 130 can include a mobile application (e.g., mobile application 220) that can activate analyte sensor 122, receive or retrieve sensor data from analyte sensor 122, and transmit sensor data from analyte sensor 122 to software application 190 (e.g., after authentication or transfer of unique identifier to remote device 130). However, for some patients, the steps needed to install the mobile app and operate it can be a barrier to use. In some aspects, when remote device 130 scans analyte sensor 122 to activate it, analyte sensor 122 can provide a URL or link for the operating system of remote device 130 to use to retrieve the mobile application (e.g., mobile application 220). In some aspects, for those patients, when the sensor is applied at a lab test facility, a mobile application (e.g., smartphone application) can be used at the lab test facility to activate the sensor and, optionally, check the functionality of the sensor if the patient waits. In some aspects, alternatively, the patient can be instructed at the lab test facility how to install and use the mobile app to check on sensor functionality and sensor life. In some aspects, the mobile app can provide a means to enter identification information and/or medication information, and transfer these to the sensor. For example, at the end of sensor wear, the patient can transfer identification information and/or medication information to analyte sensor 122 via the mobile app (e.g., remote device 130) and then mail the used sensor to the service center.

In some aspects, analyte assessment system 100 can include pharmacy options. In some aspects, in a first pharmacy option, a third party (e.g., PCP, HCP, clinician, etc.) can write a prescription for the patient, the patient can pick up the sensor “kit” at a pharmacy (e.g., including SMD 170), self-apply and activate the sensor, upload the sensor data to SMD 170 after sensor wear, and mail back SMD 170 to a service center. For example, the service center can then mail a report (e.g., glucose report) to the patient who can bring the report to their next clinical visit. In some aspects, in a second pharmacy option, a pharmacist licensed to prescribe medication (e.g., so that insurance can be billed) can advertise analyte assessment system 100 with the sensor “kit” to patients that enter their pharmacy. In some aspects, the pharmacist can apply and activate the sensor for the patient, or allow the patient to self-apply and activate the sensor at home. In some aspects, similar to the first pharmacy option, the patient can upload the sensor data to SMD 170 after sensor wear, mail back SMD 170 to a service center, and the service center can then mail a report (e.g., glucose report) to the patient who can bring the report to their next clinical visit.

Remote server 180 can be configured to provide data management, data analysis, and/or data communication with one or more components of analyte assessment system 100 (e.g., OBU 120, remote device 130, SMD 170, software application 190, etc.). Remote server 180 can be configured to support remote device 130 and/or software application 190. As shown in FIG. 1, remote server 180 can be operatively (e.g., wirelessly) coupled to remote device 130 and software application 190. In some aspects, remote server 180 can include a personal computer (e.g., smartphone), a laptop computer, an external server, a server terminal, a cloud server, a web server, or other suitable server that provides functionality for other programs and/or devices.

In some aspects, remote server 180 can be connected to a wireless network (e.g., Internet), a local area network (LAN), a wide area network (WAN), or any other data network for unidirectional or bidirectional data communication between one or more components of analyte assessment system 100 (e.g., OBU 120, remote device 130, SMD 170, software application 190, etc.). In some aspects, remote server 180 can provide new software and/or software updates (e.g., versions, patches, fixes, updates, upgrades, etc.) to one or more components of analyte assessment system 100 (e.g., OBU 120, remote device 130, SMD 170, software application 190, etc.). In some aspects, all of software application 190 can be contained in remote server 180. In some aspects, some or part of software application 190 can be contained in remote server 180, for example, supporting processing, communication, and/or reporting functionalities of software application 190.

Software application 190 can be configured to streamline requests and delivery of analyte reports (e.g., glucose reports) for third parties (e.g., PCPs, HCPs, clinicians, etc.). Software application 190 can be further configured to increase security and integrity of patient data. Software application 190 can be further configured to receive and/or store an order for analyte assessment associated with an EMR of a patient, for example, from a third party (e.g., PCP, HCP, clinician, etc.). Software application 190 can be further configured to generate or retrieve a unique identifier associated with the order. Software application 190 can be further configured to transfer the unique identifier to remote device 130 and/or SMD 170. Software application 190 can be further configured to authenticate the unique identifier with remote device 130 and/or SMD 170 (e.g., verification based on a comparison or matching of the unique identifier with patient information, analyte sensor serial number, QR sticker, or a combination thereof). Software application 190 can be further configured to activate analyte measurement system 110 (e.g., activate analyte sensor 122) based on the unique identifier. Software application 190 can be further configured to retrieve and store sensor data (e.g., of analyte sensor 122) from remote device 130 based on the order. Software application 190 can be further configured to generate a report based on the sensor data. Software application 190 can be further configured to upload the report to the EMR of the patient.

As shown in FIG. 1, software application 190 can be operatively coupled to OBU 120, remote device 130, SMD 170, and/or remote server 180. In some aspects, software application 190 can include one or more processors (e.g., processor, controller, microprocessor, microcontroller, ASIC, etc.). In some aspects, software application 190 can include and/or be coupled to a memory storing instructions, for example, instructions that when executed cause one or more processors of software application 190 to, including but not limited to, receive and store an order for analyte assessment associated with an EMR of a patient, generate or retrieve a unique identifier associated with the order, transfer the unique identifier to remote device 130 and/or SMD 170 or authenticate the unique identifier with remote device 130 and/or SMD 170 (e.g., verification based on a comparison or matching of the unique identifier with patient information, analyte sensor serial number, QR sticker, or a combination thereof), activate the analyte measurement system 110 (e.g., activate analyte sensor 122) based on the unique identifier, retrieve and store sensor data (e.g., of analyte sensor 122) from remote device 130 based on the order, generate a report based on the sensor data, and upload the report to the EMR of the patient.

In some aspects, software application 190 can be part of remote device 130. In some aspects, software application 190 can be part of SMD 170. In some aspects, software application 190 can be part of a remote device (e.g., a computer, reader, etc.), for example, at a remote location (e.g., lab test facility, service center, etc.). In some aspects, software application 190 can include a mobile application (app). For example, software application 190 can be part of remote device 130 (e.g., in a mobile app). In some aspects, software application 190 can include a web application (app). For example, software application 190 can be part of a remove device (e.g., in a web app), for example, at a remote location (e.g., lab test facility, service center, etc.).

In some aspects, remote server 180 can be configured to support all or part of software application 190. In some aspects, software application 190 can all be contained in a mobile application (app). In some aspects, some or part of software application 190 can be contained in remote server 180 (e.g., web-server, cloud server, etc.) that supports software application 190. For example, remote server 180 can support processing, communication, and/or reporting functionalities of software application 190. In some aspects, software application 190 can include an application programming interface (API) for two or more computer programs to communicate with each other.

In some aspects, processing and functionality required for software application 190 can all be contained in a mobile app. In some aspects, some or part of software application 190 (e.g., mobile app) can be contained in remote server 180 that supports software application 190, for example, remote server 180 can support the mobile app with processing, communication hub, and/or reporting functionality. In some aspects, functionality described herein for software application 190 includes functionality on the mobile app, remote server 180, or both, noting that all or some functionality can be in either or both. In some aspects, reference to software application 190 described herein implies both a mobile app and a web server (e.g., remote server 180) supporting the mobile app.

In some aspects, software application 190 (e.g., mobile app) can retrieve continuous glucose sensor data in real-time. In some aspects, software application 190 can retrieve various combinations of discrete and continuous analyte measurements. In some aspects, software application 190 can include a mechanism (e.g., algorithm) to retrieve discrete analyte measurements during a sensing period or a situation when continuous analyte measurements are not available. In some aspects, software application 190 (e.g., mobile app) can retrieve and process sensor data from OBU 120 after a custom duration (e.g., 3 days, 7 days, 14 days, 30 days, etc.), for example, after sensor wear (e.g., expiration period).

In some aspects, communication between remote device 130 and software application 190 can be NFC (e.g., limited to only NFC). In some aspects, communication between SMD 170 and software application 190 can be NFC (e.g., limited to only NFC). In some aspects, software application 190 can be configured to activate analyte measurement system 110 (e.g., analyte sensor 122), for example, through remote device 130 and/or SMD 170, based on a unique identifier. In some aspects, analyte measurement system 110 (e.g., analyte sensor 122) can be configured to store the unique identifier. In some aspects, the unique identifier can include a GUID, a UUID, a unique text string, an analyte sensor serial number, patient information, an identification sticker, a QR code, a 2D barcode, or a combination thereof. In some aspects, software application 190 can be configured to activate a professional blinded mode (e.g., in analyte sensor 122, remote device 130, and/or SMD 170) based on a unique identifier (e.g., GUID, UUID, etc.) such that sensor data is not displayed to the patient.

In some aspects, software application 190 can be located a remote location (e.g., remote to the patient), for example, a laboratory test facility or a service center. In some aspects, software application 190 can be in communication with an order interface (e.g., order interface 254) configured to receive an order for assessment of a patient from a third party (e.g., PCP, HCP, clinician, etc.). In some aspects, software application 190 can be in communication with a result interface (e.g., result interface 256) configured to provide a report (e.g., glucose report) to the third party. In some aspects, software application 190 can be in communication with a database (e.g., order database 252) configured to store the order, for example, located at a lab test facility (e.g., lab test facility 250). In some aspects, software application 190 can be in communication with a data server (e.g., remote server 280) configured to store the report.

### Exemplary EMR Assessment System

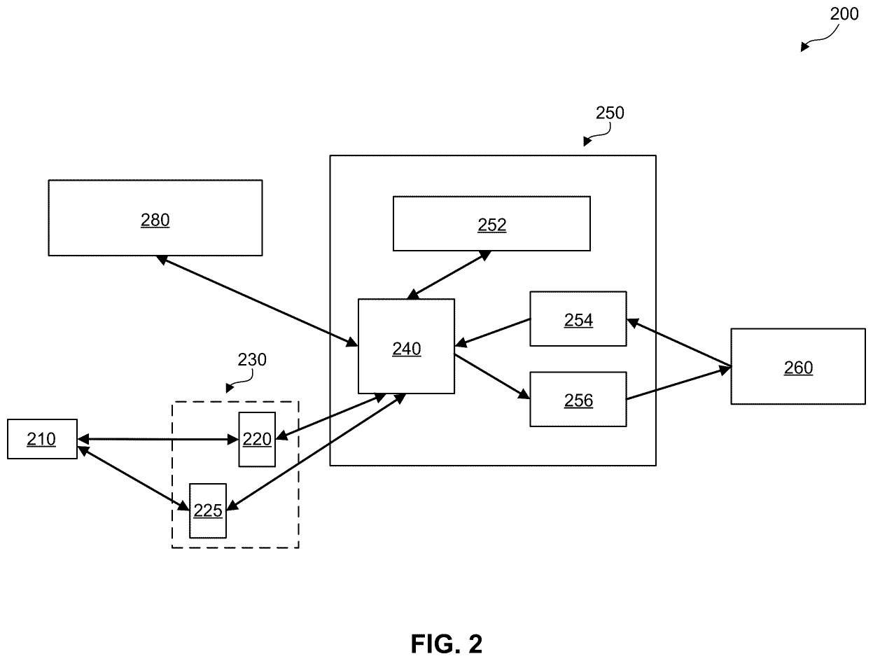

FIG. 2 illustrates EMR assessment system 200, according to exemplary aspects. EMR assessment system 200 can be configured to streamline requests and delivery of analyte reports (e.g., glucose reports) for third parties (e.g., PCPs, HCPs, clinicians, etc.) and reduce in-person patient interactions and time delays (e.g., at clinics, lab test facilities, etc.). EMR assessment system 200 can be further configured to reduce third party effort and update an EMR of the patient (e.g., similar to lab test results). EMR assessment system 200 can be further configured to increase security and integrity of patient data and operate in a blinded mode for unbiased patient analyte assessment. Although EMR assessment system 200 is shown in FIG. 2 as a stand-alone apparatus and/or system, aspects of this disclosure can be used with other apparatuses, systems, and/or methods, for example, analyte assessment system 100, clinical trial assessment system 300, flow diagram 400, and/or flow diagram 500.

The aspects of analyte assessment system 100 shown in FIG. 1, for example, and the aspects of EMR assessment system 200 shown in FIG. 2 may be similar. Similar reference numbers are used to indicate features of the aspects of analyte assessment system 100 shown in FIG. 1 and the similar features of the aspects of EMR assessment system 200 shown in FIG. 2. One difference between the aspects of analyte assessment system 100 shown in FIG. 1 and the aspects of EMR assessment system 200 shown in FIG. 2 is that remote device 230 includes mobile application (app) 220 to retrieve and transfer sensor data to software application 240 at lab test facility 250, which is coupled to order database 252, order interface 254, and result interface 256, rather than SMD 170 and software application 190 shown in FIG. 1.

In some aspects, EMR assessment system 200 can include a sensor, sensor electronics, and inserter assembly (e.g., analyte measurement system 210) and a remote device (e.g., remote device 230). In some aspects, EMR assessment system 200 can exclude a sensor management device (e.g., SMD 170) and instead use a generic device, which will be referred to herein as a “remote device,” that can include any device that can communicate with the sensor. For example, the remote device (e.g., remote device 230) can include a smartphone or tablet in conjunction with an app (e.g., mobile app 220) configured to communicate with the sensor.

In some aspects, the sensor can function in a personal mode and can be configured to do so at activation. For example, in personal mode, the sensor can function like current on-market sensors (e.g., FreeStyle Libre CGM, etc.), specifically, sending glucose values and trend information for display in near-real-time to the patient, so the patient can use this information to help control their glucose levels. In some aspects, the sensor can function in a professional mode and can be configured to do so at activation. For example, in professional mode, the sensor can restrict access to glucose data (e.g., blinded mode). In some aspects, in professional mode, the sensor can be configured to store 14 days or more (e.g., 15 days, 30 days, etc.) of glucose readings, and the glucose reading are not displayed to the patient (e.g., blinded mode).

In some aspects, EMR assessment system 200 can operate in a blinded mode to restrict access of the sensor data (e.g., glucose data) to the patient. Blinded mode is important for the glycemic assessment process because if the patient can see the readings, the patient may alter their behavior in order to correct out-of-range glucose values, which can alter the glycemic assessment and prevent the third party (e.g., PCP, HCP, clinician, etc.) from understanding the patient's glycemic condition when the patient is not using a sensor. For instance, if the professional mode was unblinded, then a patient who normally experiences hypoglycemia when not using a glucose sensor may now treat their low glucose values when they occur, the resulting glucose report (e.g., an ambulatory glucose profile (AGP)) may not indicate patterns of low glucose, and the third party (e.g., HCP) may conclude that no therapy modification is necessary. Further, when the patient stops wearing a sensor, the hypoglycemia patterns may return. In some aspects, alternatively, if the patient wears a sensor activated in a professional blinded mode, the patterns of low glucose can be recorded and reported to the third party (e.g., HCP) and the HCP can intervene in the patient's therapy, for example, reducing the amount of insulin the patient is taking during the time-of-day period when patterns of low glucose occur.

In some aspects, sensor electronics of analyte measurement system 210 (e.g., similar to on body electronics 124) can include a microcontroller or a microprocessor that can be programmed to make real-time glucose readings, store the results, and transmit the results to a remote device (e.g., remote device 230). In some aspects, sensor programming can be configured to provide a means for a remote device (e.g., remote device 230) to configure the sensor to operate in a personal mode or a professional mode. For example, sensor programming can be done by a specific or separate configuration command.

In some aspects, sensor programming can be incorporated into a sensor activation command required to initiate glucose measurements and active a sensor function (e.g., professional mode). For example, the activation command can include a variable or unique identifier that indicates the intended mode to configure the sensor (e.g., professional mode). In some aspects, sensor configuration can be made during activation. In some aspects, sensor configuration can be implemented by multiple commands. In some aspects, the activation command can include instructions to configure one or more parameters of the sensor data acquisition, for example, duration, sampling frequency, logging frequency, and/or glucose calculation type. For example, the sensor can be configured to log glucose data every 10 minutes for 15 days, where each glucose calculation can be retrospectively reprocessed to remove lag effects.

As shown in FIG. 2, EMR assessment system 200 can include analyte measurement system 210, remote device 230, software application 240, lab test facility 250, third party 260, and/or remote server 280. In some aspects, the various components of EMR assessment system 200, and the software modules that make them up (e.g., remote device 230 with mobile app 220 and/or second mobile app 225, software application 240, etc.), can be implemented on a single computer, multiple computers, on multiple web-servers, and/or on intranet servers.

Analyte measurement system 210 can be configured to measure one or more analytes (e.g., glucose) of a patient. Analyte measurement system 210 can be further configured to transfer (e.g., via remote device 230) sensor data of measured levels of an analyte (e.g., glucose). The aspects of analyte measurement system 110 shown in FIG. 1, for example, and the aspects of analyte measurement system 210 shown in FIG. 2 may be similar. Similar reference numbers are used to indicate features of the aspects of analyte measurement system 110 shown in FIG. 1 and the similar features of the aspects of analyte measurement system 210 shown in FIG. 2. In some aspects, analyte measurement system 210 can be similar to analyte measurement system 110 and analyte measurement system 210 can include insertion device 128 and OBU 120, with analyte sensor 122 and on body electronics 124. In some aspects, analyte measurement system 210 can include analyte sensor 122, which will be referred to herein as a “sensor.”

Remote device 230 can be configured to receive or retrieve sensor data from analyte measurement system 210. Remote device 230 can be further configured to activate analyte measurement system 210 (e.g., activate analyte sensor 122). Remote device 230 can be further configured to transmit sensor data from analyte measurement system 210 (e.g., analyte sensor 122) to software application 240 (e.g., after authentication or transfer of unique identifier to remote device 230). Remote device 230 can be configured to activate analyte measurement system 210 in a professional blinded mode for unbiased patient analyte assessment. The aspects of remote device 130 shown in FIG. 1, for example, and the aspects of remote device 230 shown in FIG. 2 may be similar. Similar reference numbers are used to indicate features of the aspects of remote device 130 shown in FIG. 1 and the similar features of the aspects of remote device 230 shown in FIG. 2.

As shown in FIG. 2, remote device 230 can be operatively (e.g., wirelessly, NFC, BLE, etc.) coupled to analyte measurement system 210 and software application 240. In some aspects, remote device 230 can include a handheld computer (e.g., smartphone, cell phone, mobile phone, PDA, smart watch, etc.), personal computer, laptop computer, or any other portable communication device. In some aspects, communication between analyte measurement system 210 and remote device 230 can be limited to only NFC. As shown in FIG. 2, remote device 230 can include mobile application (app) 220 and/or second mobile application (app) 225. In some aspects, mobile app 220 and second mobile app 225 can be installed and functioning on remote device 230 (e.g., a smartphone). In some aspects, alternatively, mobile app 220 and second mobile app 225 can each be implemented as dedicated electronic devices (e.g., similar to remote device 130 and/or SMD 170).

Mobile app 220 can be configured to activate analyte measurement system 210 (e.g., activate analyte sensor 122), receive or retrieve sensor data from analyte measurement system 210, and transmit sensor data from analyte measurement system 210 (e.g., analyte sensor 122) to software application 240 (e.g., after authentication or transfer of unique identifier to mobile app 220). Mobile app 220 can be further configured to activate analyte measurement system 210 (e.g., analyte sensor 122) in a professional blinded mode for unbiased patient analyte assessment. In some aspects, mobile app 220 can be installed on one or more computing devices (e.g., smartphone, tablet, computer, etc.) colocated at lab test facility 250. In some aspects, alternatively, mobile app 220 can be colocated on a computing device (e.g., tablet, reader, etc.) with software application 240.

Second mobile app 225 can be configured to monitor a functionality of analyte measurement system 210 (e.g., functionality of analyte sensor 122). In some aspects, second mobile app 225 can be configured to provide a notification to the patient (e.g., indicating measurement of sensor data is complete, indicating analyte sensor 122 expiration time, indicating sensor data error, indicating analyte sensor 122 error, etc.). In some aspects, second mobile app 225 (optional) can be installed on a patient's phone (e.g., remote device 230) and can be configured to provide the patient with the ability to monitor the function of their sensor (e.g., analyte measurement system 210). In some aspects, EMR assessment system 200 can omit second mobile app 225.

In some aspects, remote device 230 (e.g., mobile app 220) can send a unique identifier (e.g., GUID) embedded in an activation command to activate the sensor and configure the sensor to operate in professional mode. In some aspects, the sensor can store this unique identifier (e.g., GUID), for example, in its memory. In some aspects, the sensor may respond only to commands containing a unique identifier (e.g., GUID). For example, when a valid command is received by the sensor, the sensor software (e.g., on body electronics 124) can compare the GUID contained in the command with one stored in the memory of the sensor, and if they match, the sensor software can respond to the command. In some aspects, the sensor will only respond to a command to retrieve sensor data (e.g., glucose data) when the GUIDs match. In some aspects, other commands, such as sensor function or troubleshooting commands, can respond without the GUID match. In some aspects, the sensor will only respond to other commands, such as sensor function or troubleshooting commands, when the GUIDs match. In some aspects, variables other than a GUID can be used, for example, patient information (e.g., a patient's name, a patient's date-of-birth, combination thereof, etc.).

In some aspects, the sensor can store a unique identifier (e.g., GUID) and provide a command to retrieve it, but not restrict access to data based on it. In some aspects, other patient and/or clinic information can be stored on the sensor as well. In some aspects, sensor data from analyte measurement system 210 can be collocated with a unique identifier (e.g., GUID) so that the sensor data does not get erroneously associated with the wrong patient, thereby increasing security and integrity of patient data. In some aspects, the command to retrieve the data can also provide the unique identifier (e.g., GUID) and other information (e.g., patient information) so they are part of the same data packet.

Software application 240 can be configured to streamline requests and delivery of analyte reports (e.g., glucose reports) for third parties (e.g., PCPs, HCPs, clinicians, etc.). Software application 240 can be further configured to increase security and integrity of patient data. Software application 240 can be further configured to receive and/or store an order for analyte assessment associated with an EMR of a patient, for example, from a third party (e.g., PCP, HCP, clinician, etc.). Software application 240 can be further configured to generate or retrieve a unique identifier associated with the order. Software application 240 can be further configured to transfer the unique identifier to remote device 230. Software application 240 can be further configured to authenticate the unique identifier with remote device 230 (e.g., verification based on a comparison or matching of the unique identifier with patient information, analyte sensor serial number, QR sticker, or a combination thereof). Software application 240 can be further configured to activate analyte measurement system 210 (e.g., activate analyte sensor 122) based on the unique identifier. Software application 240 can be further configured to retrieve and store sensor data (e.g., of analyte sensor 122) from remote device 230 based on the order. Software application 240 can be further configured to generate a report based on the sensor data. Software application 240 can be further configured to upload the report to the EMR of the patient. The aspects of software application 190 shown in FIG. 1, for example, and the aspects of software application 240 shown in FIG. 2 may be similar. Similar reference numbers are used to indicate features of the aspects of software application 190 shown in FIG. 1 and the similar features of the aspects of software application 240 shown in FIG. 2.

As shown in FIG. 2, software application 240 can be operatively coupled to remote device 230 (e.g., mobile app 220), order database 252, order interface 254, result interface 256, and/or remote server 280. In some aspects, software application 240 can interface with each of remote device 230 (e.g., mobile app 220), order database 252, order interface 254, result interface 256, and/or remote server 280. In some aspects, software application 240 can include one or more processors (e.g., processor, controller, microprocessor, microcontroller, ASIC, etc.).

In some aspects, software application 240 can include and/or be coupled to a memory storing instructions, for example, instructions that when executed cause one or more processors of software application 240 to, including but not limited to, receive and store an order for analyte assessment associated with an EMR of a patient, generate or retrieve a unique identifier associated with the order, transfer the unique identifier to remote device 230 or authenticate the unique identifier with remote device 230 (e.g., verification based on a comparison or matching of the unique identifier with patient information, analyte sensor serial number, QR sticker, or a combination thereof), activate the analyte measurement system 210 (e.g., activate analyte sensor 122) based on the unique identifier, retrieve and store sensor data (e.g., of analyte sensor 122) from remote device 230 based on the order, generate a report based on the sensor data, and upload the report to the EMR of the patient.

In some aspects, software application 240 (e.g., mobile app) can retrieve continuous glucose sensor data in real-time. In some aspects, software application 240 can retrieve various combinations of discrete and continuous analyte measurements. In some aspects, software application 240 can include a mechanism (e.g., algorithm) to retrieve discrete analyte measurements during a sensing period or a situation when continuous analyte measurements are not available. In some aspects, software application 240 (e.g., mobile app) can retrieve and process sensor data from analyte measurement system 210 (e.g., OBU 120) after a custom duration (e.g., 3 days, 7 days, 14 days, 30 days, etc.), for example, after sensor wear (e.g., expiration period).

In some aspects, communication between remote device 230 (e.g., mobile app 220) and software application 240 can be NFC (e.g., limited to only NFC). In some aspects, communication between remote device 230 (e.g., mobile app 220) and software application 240 can be BLE (e.g., limited to only BLE). In some aspects, software application 240 can be configured to activate analyte measurement system 210 (e.g., analyte sensor 122), for example, through remote device 230 (e.g., mobile app 220), based on a unique identifier. In some aspects, analyte measurement system 210 (e.g., analyte sensor 122) can be configured to store the unique identifier. In some aspects, the unique identifier can include a GUID, a UUID, a unique text string, an analyte sensor serial number, patient information, an identification sticker, a QR code, a 2D barcode, or a combination thereof. In some aspects, software application 240 can be configured to activate a professional blinded mode (e.g., in analyte sensor 122 of analyte measurement system 210) based on a unique identifier (e.g., GUID, UUID, etc.) such that sensor data is not displayed to the patient.

In some aspects, software application 240 can be located a remote location (e.g., remote to the patient), for example, at lab test facility 250. In some aspects, software application 240 can be in communication with order interface 254 and be configured to receive an order for assessment of a patient from a third party (e.g., PCP, HCP, clinician, etc.). In some aspects, software application 240 can be in communication with result interface 256 and be configured to provide a report (e.g., glucose report) to the third party. In some aspects, software application 240 can be in communication with order database 252 and be configured to store the order, for example, located at lab test facility 250. In some aspects, software application 240 can be in communication with remote server 280 and be configured to store the report.

In some aspects, software application 240 can be used by lab test facility 250. In some aspects, software application 240 can receive one or more orders for analyte assessment (e.g., glycemic assessment) from order interface 254, for example, from third party 260 (e.g., PCP, HCP, clinician, etc.). In some aspects, software application 240 can be configured to receive and store the order. In some aspects, software application 240 can be configured to forward the order from order interface 254 to order database 252 for storage. In some aspects, order interface 254 can be configured to provide order information, including but not limited to, patient identification information, clinic and clinician identification information, patient medical information, date of the order, timestamp of the order, instructions pertaining to the order, or a combination thereof.

In some aspects, software application 240 can include a display and/or user interface (UI). For example, software application 240 can display a summary of the order on a dashboard, which can provide UI means (e.g., button, actuator, touch screen, command line, etc.) to initiate activation of the patient's sensor. In some aspects, the computing device (e.g., tablet, computer, etc.) displaying the UI of software application 240 can interface with mobile app 220 of remote device 130, for example, through a wired means (e.g., USB port, mini-USB port, serial port, Ethernet port, Internet port, etc.) or wireless means (e.g., NFC, WiFi, Bluetooth, BLE, Internet, etc.) common to device-computer communication.

In some aspects, software application 240 can generate or retrieve a new unique identifier (e.g., GUID) and sends it to mobile app 220. In some aspects, when a unique identifier (e.g., GUID) is received by mobile app 220, it enables a UI means (e.g., button, actuator, touch screen, command line, etc.) to initiate activation of the sensor and configuration of the sensor in professional mode. In some aspects, if the command(s) sent from mobile app 220 to the sensor are properly received by the sensor, and the sensor is properly configured and activated, the sensor can respond with a confirmation. For example, when mobile app 220 receives this confirmation, it can store or forward the unique identifier (e.g., GUID) associated with the order to software application 240, for example, software application 240 can forward the unique identifier (e.g., GUID) to order database 252 for storage.

In some aspects, software application 240 can receive and store an order for analyte assessment (e.g., CGM). In some aspects, software application 240 can generate a unique identifier (e.g., GUID) and store the unique identifier with the order information. In some aspects, mobile app 220 can provide a means for a user to retrieve the order based on patient information. For example, mobile app 220 can send patient information to software application 240, software application 240 can search active orders for patient information, and when a match is found software application 240 can send the associated GUID to mobile app 220.

In some aspects, mobile app 220 can provide a means to activate a sensor. For example, mobile app 220 can include a unique identifier (e.g., GUID) with the activation command to activate the sensor and the sensor can store the unique identifier (e.g., GUID). In some aspects, mobile app 220 can provide a means to upload sensor data (e.g., glucose data) from the sensor. For example, the sensor can respond to an upload command (e.g., with the unique identifier) from mobile app 220 by sending the stored sensor data and the unique identifier (e.g., GUID) to mobile app 220.

In some aspects, mobile app 220 can transfer the sensor data (e.g., glucose data) and associated unique identifier (e.g., GUID) to software application 240. In some aspects, software application 240 can search order database 252 of active orders for a match to the uploaded unique identifier (e.g., GUID). In some aspects, software application 240 can store the sensor data and associate the sensor data with the corresponding order information. In some aspects, software application 240 can generate one or more reports from the stored sensor data (e.g., glucose data). In some aspects, software application 240 can upload these reports via result interface 256 to the EMR account specified in the associated order.

In some aspects, EMR assessment system 200 can perform one or more of the following steps: (1) software application 240 can receive and store an order for CGM assessment; (2) software application 240 can generate a GUID and store the GUID with the order information; (3) mobile app 220 can provide a means for a user to retrieve the order based on patient information—(3a) mobile app 220 can send patient information to software application 240, and (3b) software application 240 can search active orders for patient information, and when a match is found, send the associated GUID to mobile app 220; (4) mobile app 220 can provide a means to activate a sensor; (5) mobile app 220 can include the GUID with a command to activate the sensor; (6) the sensor can store the GUID; (7) mobile app 220 can provide a means to upload glucose data from the sensor; (8) the sensor can respond to an upload command by sending the stored glucose data and GUID to mobile app 220; (9) mobile app 220 can transfer the glucose data and associated GUID to software application 240; (10) software application 240 can search the database of active orders for a match to the uploaded GUID; (11) software application 240 can store and associate the glucose data with the order information; (12) software application 240 can generate reports from the stored glucose data; and (13) software application 240 can upload these reports to the EMR account specified in the associated order.

In some aspects, alternatively, software application 240, mobile app 220, and the sensor (e.g., analyte measurement system 210) can be configured such that the sensor provides its serial number upon activation to mobile app 220, and mobile app 220 then sends the sensor serial number back to software application 240 where it is associated with the order information.

In some aspects, alternatively, software application 240, mobile app 220, and the sensor (e.g., analyte measurement system 210) can be configured such that all or some of the order information is located on the sensor (e.g., stored in the sensor's memory). For example, when the sensor data is retrieved by mobile app 220, the order information can be included with the sensor data (e.g., glucose data) in the transfer.

In some aspects, the dashboard of software application 240 can also provide a UI means (e.g., button, actuator, touch screen, command line, etc.) to enable the patient's second mobile app 225 to monitor the functionality of their sensor. For example, software application 240 can store the unique identifier (e.g., GUID) and the time of sensor activation on second mobile app 225, and second mobile app 225 can provide a UI means (e.g., button, actuator, touch screen, command line, etc.) to send the sensor (e.g., analyte measurement system 210) a command to request sensor status. In some aspects, second mobile app 225 can include a timer and an alert indicating to the patient that the sensor readings are complete. In some aspects, second mobile app 225 can include a notification to the patient when sensor readings are complete, for example, reminding the patient to mail back the used sensor back to lab test facility 250.

In some aspects, when lab test facility 250 receives the used sensor, mobile app 220 can be used to retrieve the sensor data (e.g., glucose data). In some aspects, patient identification information can be contained within a package containing the sensor, and software application 240 can be configured to provide a UI means (e.g., button, actuator, touch screen, command line, etc.) to retrieve data of a particular patient via the dashboard of software application 240. For example, when this UI means is activated by an operator, software application 240 can retrieve the unique identifier (e.g., GUID) for that particular patient and send the unique identifier (e.g., GUID) to mobile app 220, which can enable a scan UI on mobile app 220 to retrieve patient data (e.g., sensor data).

In some aspects, a UI means (e.g., button, actuator, touch screen, command line, etc.) can be provided on mobile app 220 to initiate data collection from the sensor. For example, mobile app 220 can send a data retrieval command to the sensor which includes the unique identifier (e.g., GUID), the sensor software can be configured to detect whether this unique identifier (e.g., GUID) matches the unique identifier (e.g., GUID) stored on the sensor (e.g., in sensor memory), and if there is a match, the sensor sends the sensor data (e.g., glucose data) to mobile app 220. In some aspects, once mobile app 220 receives the sensor data, mobile app 220 can then send the sensor data to software application 240, which can store or forward this data associated with the order information.

In some aspects, alternatively, software application 240, mobile app 220, and the sensor (e.g., analyte measurement system 210) can be configured such that mobile app 220 can send a command to the sensor to retrieve the sensor serial number. For example, mobile app 220 can retrieve the sensor serial number and then send the sensor serial number to software application 240, which can then retrieve a unique identifier (e.g., GUID) associated with the sensor serial number, and software application 240 can then send the unique identifier (e.g., GUID) to mobile app 220.

In some aspects, if patient identification information is not provided, software application 240 can send a series of unique identifiers (e.g., GUIDs) to mobile app 220 to attempt to download the sensor data (e.g., glucose data) from the sensor. In some aspects, alternatively, at the time of activation, software application 240 can generate a separate unique code (e.g., a QR code), which can be sent to a sticker printer, and the sticker can be affixed to a sensor mail envelope. For example, mobile app 220 can have a camera function with a QR code scan feature that can send the unique code to a server (e.g., remote server 280) where it can be retrieved by software application 240, identifying the patient and the proper unique identifier (e.g., GUID) to send to mobile app 220.

In some aspects, the dashboard of software application 240 can indicate when the sensor data (e.g., glucose data) is retrieved from the order. In some aspects, the dashboard of software application 240 can provide a UI means (e.g., button, actuator, touch screen, command line, etc.) to initiate analyte reports (e.g., glucose reports) and send these reports (e.g., via result interface 256) to the clinic EMR (e.g., third party 260) indicated in the associated order information. In some aspects, alternatively, once the sensor data (e.g., glucose data) is received by software application 240, software application 240 can automatically generate reports and automatically send the reports to the clinic EMR (e.g., third party 260).

In some aspects, when the sensor data (e.g., glucose data) is retrieved, software application 240 can forward the sensor data to a real-world data server (e.g., remote server 280). In some aspects, other information associated with the order (e.g., patient information, etc.) can be sent to software application 240 at any time. In some aspects, data can be redacted (e.g., anonymized) in order to hide the identity of the patient, for example, before being sent to the real-world data server (e.g., remote server 280). In some aspects, software application 240 and/or order database 252 can provide a patient code so that data across multiple orders can be associated together, but without providing any patient identifying information.

In some aspects, software application 240 can use sensor data (e.g., glucose data) from the sensor (e.g., analyte measurement system 210) and associated order information to generate reports (e.g., glucose reports). For example, software application 240 can generate a glucose patterns report with medication considerations based on the patient medication information and medical information provided in the order.

In some aspects, the sensor (e.g., analyte measurement system 210) can exchange information with mobile app 220 only by NFC. In some aspects, the sensor (e.g., analyte measurement system 210) can exchange information with second mobile app 225 only by NFC. In some aspects, the sensor (e.g., analyte measurement system 210) can exchange information with mobile app 220 and/or second mobile app 225 by BLE.

Lab test facility 250 can be configured to receive an order for assessment from a third party (e.g., PCP, HCP, clinician, third party 260, etc.), generate analyte report(s) associated with the order, and deliver the analyte report(s) to the third party. Lab test facility 250 can be further configured to receive and store multiple orders from one or more third parties (e.g., PCP, HCP, clinician, third party 260, etc.) in order database 252. Lab test facility 250 can be further configured to maintain software application 240 at the location. As shown in FIG. 2, lab test facility 250 can include software application 240, order database 252, order interface 254, and result interface 256. Order database 252 can be configured to store received orders from one or more third parties (e.g., PCP, HCP, clinician, third party 260, etc.) and communicate with software application 240. Order database 252 can be further configured to provide business applications for lab test facility 250 (e.g., patient information, insurance billing, CPT codes, etc.).

Order interface 254 can be configured to receive an order for assessment of a patient from a third party (e.g., PCP, HCP, clinician, third party 260, etc.). As shown in FIG. 2, order interface 254 can be in communication with software application 240 and a third party 260. In some aspects, order interface 254 can receive an order for assessment from third party 260 and send the order to software application 240, which can store the order (e.g., in memory) and/or forward the order to be stored in order database 252.

Result interface 256 can be configured to provide a report (e.g., glucose report) to the third party (e.g., PCP, HCP, clinician, third party 260, etc.) associated with the order for assessment. As shown in FIG. 2, result interface 256 can be in communication with software application 240 and a third party 260. In some aspects, result interface 256 can receive a generated report associated with the order for assessment from software application 240 and send the report to the third party 260, for example, by updated an EMR associated with the patient.

In some aspects, third party 260 can include any third party that is not the patient. For example, third party 260 can include a PCP, HCP, clinician, clinic, caregiver, research site, institution, industry sponsor, CRO, or a combination thereof.

Remote server 280 can be configured to be a real-world data server in communication with software application 240. Remote server 280 can be further configured to provide data management, data analysis, and/or data communication with one or more components of EMR assessment system 200 (e.g., analyte measurement system 210, mobile app 220, second mobile app 225, software application 240, order database 252, etc.). Remote server 280 can be further configured to store redacted (e.g., anonymized) sensor data and/or reports for big data analysis. The aspects of remote server 180 shown in FIG. 1, for example, and the aspects of remote server 280 shown in FIG. 2 may be similar. Similar reference numbers are used to indicate features of the aspects of remote server 180 shown in FIG. 1 and the similar features of the aspects of remote server 280 shown in FIG. 2.

As shown in FIG. 2, remote server 280 can be operatively (e.g., wirelessly) coupled to software application 240. In some aspects, remote server 280 can include a personal computer (e.g., smartphone), a laptop computer, an external server (e.g., real-world-data server, the Internet, etc.), a server terminal, a cloud server, a web server, or other suitable server that provides functionality and storage for other programs and/or devices. In some aspects, software application 240 can interface with remote server 280 to easily retrieve and/or store reports, for example, for generating confidential (anonymous) big data based on the reports.

### Exemplary Clinical Trial Assessment System

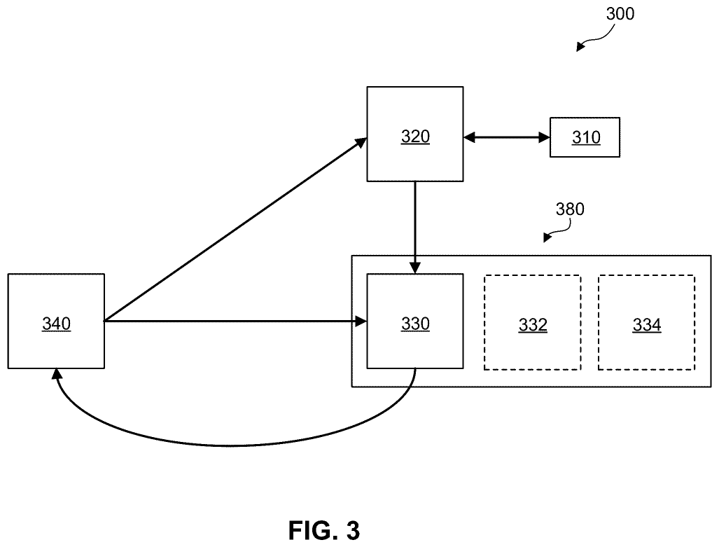

FIG. 3 illustrates clinical trial assessment system 300, according to exemplary aspects. Clinical trial assessment system 300 can be configured to initiate a clinical trial, create an independent storage server (e.g., cloud server) associated with the clinical trial, and export sensor data from multiple participants to the independent storage server. Clinical trial assessment system 300 can be further configured to streamline requests and delivery of reports (e.g., glucose reports, participant data, clinical trial metrics, etc.) for third parties (e.g., PCPs, HCPs, clinicians, research sites, institutions, industry sponsors, CROs, etc.). Clinical trial assessment system 300 can be further configured to provide role-based access control for third parties (e.g., PCPs, HCPs, clinicians, research sites, institutions, industry sponsors, CROs, etc.) to review participant data, assess therapy changes, monitor participant health and safety, and/or modify parameters of the clinical trial. Although clinical trial assessment system 300 is shown in FIG. 3 as a stand-alone apparatus and/or system, aspects of this disclosure can be used with other apparatuses, systems, and/or methods, for example, analyte assessment system 100, EMR assessment system 200, flow diagram 400, and/or flow diagram 500.

The aspects of analyte assessment system 100 shown in FIG. 1, for example, and the aspects of clinical trial assessment system 300 shown in FIG. 3 may be similar. Similar reference numbers are used to indicate features of the aspects of analyte assessment system 100 shown in FIG. 1 and the similar features of the aspects of clinical trial assessment system 300 shown in FIG. 3. One difference between the aspects of analyte assessment system 100 shown in FIG. 1 and the aspects of clinical trial assessment system 300 shown in FIG. 3 is that web application (app) 340 creates independent cloud server 330 and uses mobile application (app) 320 and independent cloud server 330 to retrieve and transfer sensor data associated with a clinical trial to web app 340, rather than SMD 170 and software application 190 shown in FIG. 1.

As shown in FIG. 3, clinical trial assessment system 300 can include analyte measurement system 310, mobile app 320, independent cloud server 330 (e.g., part of external server 380), and web app 340. In some aspects, the various components of clinical trial assessment system 300, and the software modules that make them up (e.g., mobile app 320, independent cloud server 330, web app 340, etc.), can be implemented on a single computer, multiple computers, on multiple web-servers, and/or on intranet servers.

Analyte measurement system 310 can be configured to measure one or more analytes (e.g., glucose) of a patient. Analyte measurement system 310 can be further configured to transfer (e.g., via mobile app 320) sensor data of measured levels of an analyte (e.g., glucose). The aspects of analyte measurement system 110 shown in FIG. 1, for example, and the aspects of analyte measurement system 310 shown in FIG. 3 may be similar. Similar reference numbers are used to indicate features of the aspects of analyte measurement system 110 shown in FIG. 1 and the similar features of the aspects of analyte measurement system 310 shown in FIG. 3. In some aspects, analyte measurement system 310 can be similar to analyte measurement system 110 and analyte measurement system 310 can include insertion device 128 and OBU 120, with analyte sensor 122 and on body electronics 124. In some aspects, analyte measurement system 310 can include analyte sensor 122, which will be referred to herein as a “sensor.” In some aspects, analyte measurement system 310 can include a glucose sensor (e.g., CGM) configured to measure glucose data of a patient.

Mobile app 320 can be configured to activate analyte measurement system 310 (e.g., activate analyte sensor 122), receive or retrieve sensor data from analyte measurement system 310, and transmit sensor data from analyte measurement system 310 (e.g., analyte sensor 122) to independent cloud server 330 (e.g., after authentication or transfer of unique identifier to mobile app 320). Mobile app 320 can be further configured to activate analyte measurement system 310 (e.g., analyte sensor 122) in a blinded mode for unbiased patient analyte assessment. The aspects of mobile app 220 shown in FIG. 2, for example, and the aspects of mobile app 320 shown in FIG. 3 may be similar. Similar reference numbers are used to indicate features of the aspects of mobile app 220 shown in FIG. 2 and the similar features of the aspects of mobile app 320 shown in FIG. 3.

As shown in FIG. 3, mobile app 320 can be operatively (e.g., wirelessly, NFC, BLE, etc.) coupled to analyte measurement system 310, independent cloud server 330, and web app 340. In some aspects, mobile app 320 can be installed and functioning on a handheld computer (e.g., smartphone, cell phone, mobile phone, PDA, smart watch, etc.), personal computer, laptop computer, or any other portable communication device. In some aspects, communication between analyte measurement system 310 and mobile app 320 can be limited to only NFC. In some aspects, communication between analyte measurement system 310 and mobile app 320 can be limited to only BLE. In some aspects, alternatively, mobile app 320 can be implemented as a dedicated electronic device (e.g., similar to remote device 130 and/or SMD 170).

In some aspects, mobile app 320 can be configured to activate the sensor (e.g., analyte measurement system 310) in a blinded mode such that sensor data (e.g., glucose data) is not displayed to the patient. For example, the blinded mode can provide unbiased patient analyte assessment and reduce participants altering their behavior in response to displayed analyte readings during the clinical trial. In some aspects, mobile app 320 can be configured to activate the sensor (e.g., analyte measurement system 310) in an unblinded mode such that sensor data (e.g., glucose data) is displayed to the patient. For example, during all or some of the clinical trial, participants can be shown analyte readings, for example, to increase health and safety during the clinical trial. In some aspects, mobile app 320 can be configured to collect sensor data (e.g., glucose data) over a custom duration. For example, the custom duration can be based on sensor wear (e.g., 3 days, 7 days, 14 days, 30 days, etc.), for example, 14 days. In some aspects, mobile app 320 can perform no data analysis or interpretation. In some aspects, mobile app 320 can perform continuous data communication with the sensor (e.g., analyte measurement system 310) and receive sensor data in real-time or near real-time.

In some aspects, mobile app 320 can be downloaded and installed on a participant's personal computing device (e.g., smartphone, etc.), for example, similar to remote device 130. In some aspects, mobile app 320 can include the new clinical trial parameters and guidelines. In some aspects, mobile app 320 can remotely activate a new sensor in a blinded mode. In some aspects, mobile app 320 can collect sensor data (e.g., glucose data) from the sensor over a specified duration, for example, 14 days.

Independent cloud server 330 can be configured to receive and store data (e.g., glucose data) from mobile app 320 associated with a clinical trial. As shown in FIG. 3, independent cloud server 330 can be operatively (e.g., wirelessly, etc.) coupled to mobile app 320 and web app 340. In some aspects, independent cloud server 330 can receive sensor data from multiple mobile apps 320 associated with multiple participants in the clinical trial. In some aspects, clinical trial assessment system 300 can include external server 380 configured to support one or more independent cloud servers each associated with a specific clinical trial. For example, as shown in FIG. 3, external server 380 can include independent cloud server 330, second independent cloud server 332, and/or third independent cloud server 334. In some aspects, independent cloud server 330 may be 21 CFR compliant (e.g., Title 21, Part 11 of the CFR).

In some aspects, independent cloud server 330 can be created when the new clinical trial is created, for example, by web app 340. In some aspects, participant data (e.g., country, age, name, demographic, medical history, medications, sensor usage, etc.) can be exported to independent cloud server 330 during the clinical trial. In some aspects, clinical trial metrics (e.g., operational metrics, performance metrics, KPIs, operational performance, number of KOLs, number of CROs, etc.) can be exported to independent cloud server 330 during the clinical trial.

Web app 340 can be configured to initiate a clinical trial, create independent cloud server 330 associated with the clinical trial, and export sensor data from multiple participants to independent cloud server 330. Web app 340 can be further configured to store the sensor data, generate one or more reports (e.g., glucose reports) based on the sensor data, and share the one or more reports with a third party. Web app 340 can be further configured to streamline requests and delivery of reports (e.g., glucose reports, participant data, clinical trial metrics, etc.) for third parties (e.g., PCPs, HCPs, clinicians, research sites, institutions, industry sponsors, CROs, etc.). Web app 340 can be further configured to provide role-based access control for third parties (e.g., PCPs, HCPs, clinicians, research sites, institutions, industry sponsors, CROs, etc.) to review participant data, assess therapy changes, monitor participant health and safety, and/or modify parameters of the clinical trial. The aspects of software application 240 shown in FIG. 2, for example, and the aspects of web app 340 shown in FIG. 3 may be similar. Similar reference numbers are used to indicate features of the aspects of software application 240 shown in FIG. 2 and the similar features of the aspects of web app 340 shown in FIG. 2.

In some aspects, web app 340 can be configured to generate or retrieve a unique identifier associated with the clinical trial. In some aspects, web app 340 can be configured to authenticate the unique identifier with mobile app 320 (e.g., verification based on a comparison or matching of the unique identifier with patient information, analyte sensor serial number, QR sticker, or a combination thereof). In some aspects, web app 340 can be configured to activate analyte measurement system 310 (e.g., activate analyte sensor 122) based on the unique identifier. In some aspects, web app 340 can be configured to retrieve and store sensor data (e.g., of analyte sensor 122) from mobile app 320 in independent cloud server 330. In some aspects, web app 340 can be configured to generate a report based on the sensor data.

As shown in FIG. 3, web app 340 can be operatively coupled to mobile app 320 and independent cloud server 330. In some aspects, web app 340 can interface with each of mobile app 220 and/or independent cloud server 330. In some aspects, web app 340 can include one or more processors (e.g., processor, controller, microprocessor, microcontroller, ASIC, etc.). In some aspects, web app 340 can create a new clinical trial including specific parameters and guidelines to be implemented on sensors and mobile apps 320 of multiple participants. For example, web app 340 can setup participant mobile app configuration (e.g., mobile app 320), setup number of participants and number of countries conducting the clinical trial, and/or setup specific KPIs or clinical trial metrics.

In some aspects, web app 340 can be configured to provide role-based access control to users. For example, access to different levels of data (e.g., sensor data, participant data, clinical trial metrics, etc.) can be provided (e.g., tailored) to different users, for example, based on authorization levels (e.g., participant, clinician, industry sponsor, CRO, etc.). In some aspects, web app 340 can provide confidential and secure access to different levels of data associated with the clinical trial based on the specific user (e.g., PCPs, HCPs, clinicians, research sites, institutions, industry sponsors, CROs, etc.), for example, access to data can be configured based on roles and industry sponsor requirements.

In some aspects, web app 340 can be configured to make therapy changes during the clinical trial. For example, based on sensor data and/or clinical trial metrics, a third party (e.g., PCPs, HCPs, clinicians, research sites, institutions, industry sponsors, CROs, etc.) can adjust the therapy accordingly, for example, increase or decrease an amount of a medication being using during the clinical trial. In some aspects, web app 340 can be configured to monitor clinical trial metrics during the clinical trial. For example, web app 340 can monitor countries conducting the clinical trial, research sites conducting the clinical trial, total number of participants, number of participants per country, number of participants per research site, total usage of participants, or a combination thereof.

In some aspects, web app 340 can be configured to review participant specific data (e.g., glucose reports, sensor usage, country, medical history, etc.). In some aspects, web app 340 can be configured to assess therapy changes, for example, based on clinical trial metrics (e.g., KPIs, etc.) measured throughout the clinical trial. In some aspects, web app 340 can be configured to monitor participant health and safety during the clinical trial, for example, stopping the trial if a threshold of participants continually experience adverse reactions (e.g., hypoglycemia, ketoacidosis, etc.).

In some aspects, web app 340 can be configured to run clinical trials with different study designs (e.g., clinical trial metrics). For example, web app 340 can run interventional clinical trials, observational clinical trials, or a combination thereof. In some aspects, web app 340 can be configured to run clinical trials with different blinding and/or data masking options. For example, web app 340 can activate sensors in the clinical trial in a blinded mode to prevent the participant from viewing the glucose data. In some aspects, web app 340 can be configured to minimize effort to onboard participating sites, for example, by being able to add a clinical research site to the clinical trial. In some aspects, web app 340 can provide role-based access to site users, for example, web app 340 can configure data access based on roles and sponsor requirements.

In some aspects, web app 340 can lower participant burden (e.g., reduce site visits for sensor activation) and lower site burden and cost (e.g., reduce number of visits and effort at site to activate each sensor). For example, web app 340 can provide remote or on-site sensor activation using participant's mobile app 320. In some aspects, web app 340 can streamline analysis of data for primary, secondary, and/or exploratory endpoints. For example, web app 340 (e.g., via mobile app 320 and independent cloud server 330) can collect and export historical glucose data (e.g., based on a 5 minute interval). For example, web app 340 (e.g., via mobile app 320 and independent cloud server 330) can view glucose data over a custom duration (e.g., day 3 to day 7, day 10 to day 12, etc.). In some aspects, web app 340 can utilize analytics of collected data to quickly monitor clinical trial performance. For example, web app 340 can view and monitor clinical trial metrics (e.g., operational metrics, performance metrics, KPIs, etc.).

In some aspects, web app 340 can provide a self-service setup for a clinical trial. For example, web app 340 can provide a UI means to setup the clinical trial based on business agreements, timeframes, and reduce lead time for clinical trial setup. In some aspects, web app 340 can provide editing functionality for the clinical trial. For example, web app 340 can provide a UI means to modify the clinical trial to accommodate changes in clinical trial requirements (e.g., protocol amendments, etc.). In some aspects, web app 340 can provide terminating functionality for the clinical trial. For example, web app 340 can provide a UI means to end the clinical trial and minimize effort to disable product access (e.g., mobile app 320) after the clinical trial ends. In some aspects, web app 340 can provide industry sponsor access. For example, web app 340 can provide a UI means to provide customer access (e.g., industry sponsor access) to manage sites and minimize efforts to support day-to-day operations (e.g., providing a daily summary).

In some aspects, web app 340 can set data collection requirements for one or more clinical trials. For example, web app 340 can support a range of clinical trials (e.g., types, outcome measures, etc.) for users and provide a UI means to set default sensor collection values (e.g., historical glucose data at 5 minute interval, etc.). In some aspects, web app 340 can review clinical trial metrics. For example, web app 340 can provide a UI means to review clinical trial metrics (e.g., operational metrics, performance metrics, KPIs, etc.) during the clinical trial, for example, reviewing usage of product (e.g., sensor, mobile app 320, etc.) and ensuring usage is in accordance with business agreements.

### Exemplary Sensor End of Life Scenarios

In some aspects, mobile app 320 may display the number of days remaining for analyte measurement system 310 (e.g., analyte sensor 122) to continue monitoring and sending glucose data of the user to independent cloud server 330 before it needs to be replaced (e.g., display “3 days left on sensor until end of life”). In some aspects, for example, mobile app 320 may provide an alarm or alert to the user when analyte sensor 122 is near end of life (e.g., 14 days). In some aspects, for example, mobile app 320 may provide a notification or reminder to the user to have their phone in range of analyte measurement system 310 all day for a specific day (e.g., 15^{th} day of sensor wear) when near sensor end of life (e.g., 15 days). In some aspects, mobile app 320 may provide a status update of analyte measurement system 310 regarding sensor functionality. In some aspects, for example, mobile app 320 may provide a notification or alert to the user if analyte sensor 122 is malfunctioning or outside one or more calibration thresholds.

In some aspects, mobile app 320 may confirm whether data from analyte measurement system 310 was uploaded properly to independent cloud server 330. In some aspects, for example, mobile app 320 may provide a notification or alert that the analyte measurement system 310 upload has failed and request one or more actions from the user. For example, the alert may include a lost signal alarm (e.g., no WiFi or BLE) or out of range alarm, and request that the user re-establish connection with analyte measurement system 310 (e.g., user walked away from phone). In some aspects, if analyte measurement system 310 is near end of life (e.g., 14 days), mobile app 320 may collect glucose data from analyte measurement system 310 at a higher frequency (e.g., collect glucose data every minute) to ensure data is collected before analyte measurement system 310 (e.g., analyte sensor 122) becomes inoperable.

### Exemplary Flow Diagrams

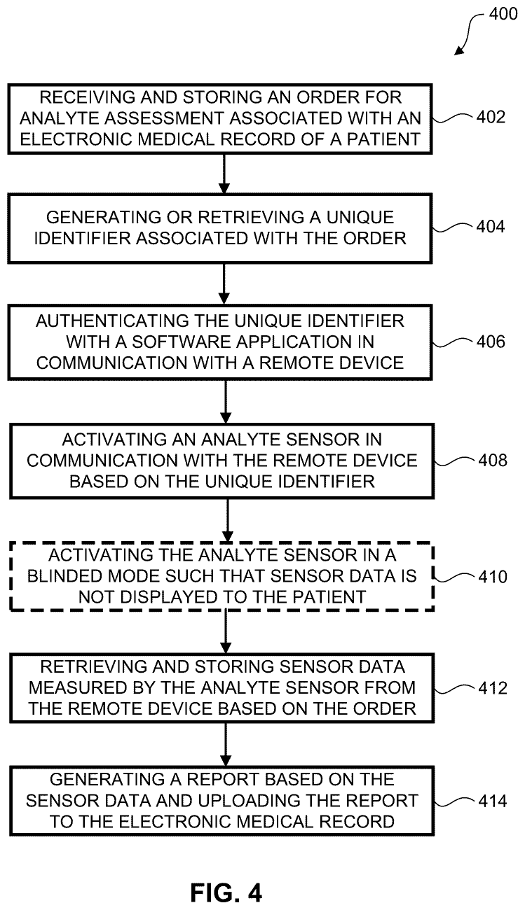

FIG. 4 illustrates flow diagram 400 for EMR assessment system 200 shown in FIG. 2, according to an exemplary aspect. Flow diagram 400 can be configured to streamline requests and delivery of analyte reports (e.g., glucose reports) for third parties (e.g., PCPs, HCPs, clinicians, etc.) and reduce in-person patient interactions and time delays (e.g., at clinics, lab test facilities, etc.). Flow diagram 400 can be further configured to reduce third party effort and update an EMR of the patient (e.g., similar to lab test results). Flow diagram 400 can be further configured to increase security and integrity of patient data, and, optionally, operate in a blinded mode for unbiased patient analyte assessment. It is to be appreciated that not all steps in FIG. 4 are needed to perform the disclosure provided herein. Further, some of the steps may be performed simultaneously, sequentially, and/or in a different order than shown in FIG. 4. Flow diagram 400 shall be described with reference to FIGS. 1 and 2. However, flow diagram 400 is not limited to those example aspects. Although flow diagram 400 is shown in FIG. 4 as a stand-alone method, aspects of this disclosure can be used with other apparatuses, systems, and/or methods, for example, analyte assessment system 100, EMR assessment system 200, clinical trial assessment system 300, and/or flow diagram 500. In some aspects, flow diagram 400 can be implemented by analyte assessment system 100 shown in FIG. 1. In some aspects, flow diagram 400 can be implemented by EMR assessment system 200 shown in FIG. 2.

In step 402, as shown in the example of FIG. 2, an order for analyte assessment, associated with an EMR of a patient, can be sent by a third party 260 to lab test facility 250 via order interface 254, which can be received and stored by software application 240. In some aspects, software application 240 can receive and forward the order to order database 252 of lab test facility 250 for storage.

In step 404, as shown in the example of FIG. 2, a unique identifier (e.g., GUID) associated with the order can be generated or retrieved by software application 240. In some aspects, software application 240 can generate the unique identifier (e.g., GUID) associated with the order, for example, software application 240 can generate a GUID (e.g., 128-bit unique text string generator) and store the GUID with the order information. In some aspects, software application 240 can retrieve the unique identifier (e.g., GUID) associated with the order, for example, software application 240 (e.g., via mobile app 220) can retrieve the sensor's serial number associated with the order.

In step 406, as shown in the example of FIG. 2, software application 240 can authenticate the unique identifier (e.g., GUID) with remote device 230 (e.g., via mobile app 220). In some aspects, authenticating the unique identifier (e.g., GUID) can include mobile app 220 sending patient information to software application 240, software application 240 searching active orders for patient information, and when a match is found, sending the associated unique identifier (e.g., GUID) to mobile app 220. In some aspects, authenticating the unique identifier (e.g., GUID) can include comparing or matching of the unique identifier with patient information, analyte sensor serial number, QR sticker, or a combination thereof to confirm or validate the unique identifier (e.g., GUID) is correct.

In step 408, as shown in the example of FIG. 2, mobile app 220 can activate a sensor (e.g., analyte measurement system 210) based on the unique identifier. In some aspects, mobile app 220 can include the unique identifier (e.g., GUID) with a command to activate the sensor.

In step 410, optionally, as shown in the example of FIG. 2, mobile app 220 can activate the sensor in a blinded mode such that sensor data is not displayed to the patient. In some aspects, communication between the sensor (e.g., analyte measurement system 210) and mobile app 220 can be limited to only NFC.

In step 412, as shown in the example of FIG. 2, software application 240 can retrieve and store sensor data of the sensor from mobile app 220 based on the order. In some aspects, the sensor can respond to an upload command from mobile app 220 by sending the stored sensor data and unique identifier (e.g., GUID) to mobile app 220, mobile app 220 can transfer the sensor data and associated unique identifier (e.g., GUID) to software application 240, software application 240 can search the database of active orders for a match to the uploaded unique identifier (e.g., GUID), and software application 240 can store and associate the sensor data with the order information.

In step 414, as shown in the example of FIG. 2, software application 240 can generate a report based on the sensor data, and upload the report to the EMR of the patient. In some aspects, software application 240 can generate reports from the stored sensor data, and software application 240 can upload these reports (e.g., via result interface 256) to the EMR account specified in the associated order (e.g., to third party 260).

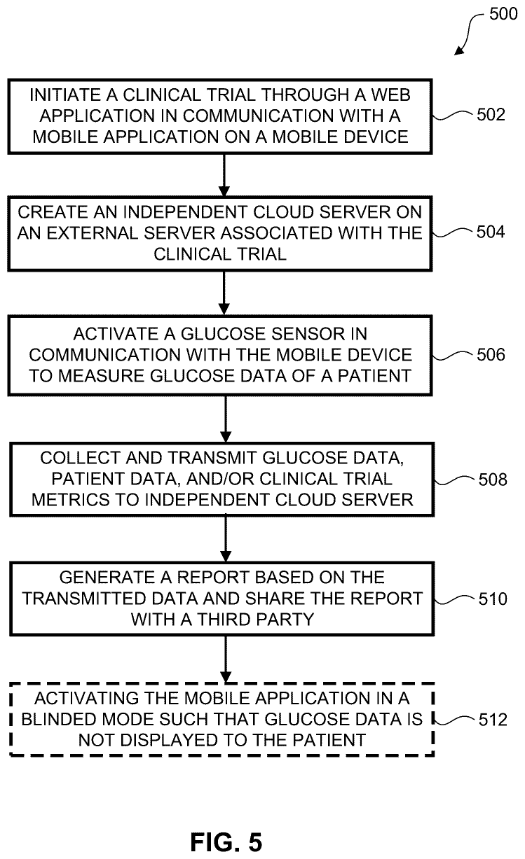

FIG. 5 illustrates flow diagram 500 for clinical trial assessment system 300 shown in FIG. 3, according to an exemplary aspect. Flow diagram 500 can be configured to initiate a clinical trial, create an independent storage server (e.g., cloud server) associated with the clinical trial, and export sensor data from multiple participants to the independent storage server. Flow diagram 500 can be further configured to streamline requests and delivery of reports (e.g., glucose reports, participant data, clinical trial metrics, etc.) for third parties (e.g., PCPs, HCPs, clinicians, research sites, institutions, industry sponsors, CROs, etc.). Flow diagram 500 can be further configured to provide role-based access control for third parties (e.g., PCPs, HCPs, clinicians, research sites, institutions, industry sponsors, CROs, etc.) to review participant data, assess therapy changes, monitor participant health and safety, and/or modify parameters of the clinical trial. It is to be appreciated that not all steps in FIG. 5 are needed to perform the disclosure provided herein. Further, some of the steps may be performed simultaneously, sequentially, and/or in a different order than shown in FIG. 5. Flow diagram 500 shall be described with reference to FIGS. 1 and 3. However, flow diagram 500 is not limited to those example aspects. Although flow diagram 500 is shown in FIG. 5 as a stand-alone method, aspects of this disclosure can be used with other apparatuses, systems, and/or methods, for example, analyte assessment system 100, EMR assessment system 200, clinical trial assessment system 300, and/or flow diagram 400. In some aspects, flow diagram 500 can be implemented by analyte assessment system 100 shown in FIG. 1. In some aspects, flow diagram 500 can be implemented by clinical trial assessment system 300 shown in FIG. 3.

In step 502, as shown in the example of FIG. 3, web app 340 can initiate a clinical trial in communication with one or more mobile apps 320 on one or more corresponding mobile devices (e.g., participant's smartphone). In some aspects, web app 340 can provide a UI means to create (e.g., setup) a new clinical trial including specific parameters and guidelines to be implemented on sensors and mobile apps 320 of multiple participants, for example, web app 340 can setup participant mobile app configurations (e.g., mobile app 320), data collection requirements (e.g., historical glucose data at 5 minute intervals), the number of participants in the clinical trial, and/or specific KPIs or clinical trial metrics.

In step 504, as shown in the example of FIG. 3, web app 340 can create an independent cloud server 330 on an external server 380 associated with the clinical trial. In some aspects, independent cloud server 330 can receive and store sensor data (e.g., glucose data) from mobile app 320, participant data, and/or clinical trial metrics associated with a clinical trial. In some aspects, independent cloud server 330 can receive sensor data from multiple mobile apps 320 associated with multiple participants in the clinical trial.

In step 506, as shown in the example of FIG. 3, mobile app 320 can activate a corresponding sensor (e.g., analyte measurement system 310) to measure sensor data (e.g., glucose data) of a participant. In some aspects, mobile app 220 can activate the sensor based on a unique identifier, for example, mobile app 220 can include the unique identifier (e.g., GUID) with a command to activate the sensor.

In step 508, as shown in the example of FIG. 3, mobile app 320 can collect and transmit sensor data (e.g., glucose data), participant data, and/or clinical trial metrics to independent cloud server 330. In some aspects, the sensor can respond to an upload command from mobile app 320 by sending the stored sensor data and a unique identifier (e.g., GUID) to mobile app 320, mobile app 320 can transfer the sensor data and associated unique identifier (e.g., GUID) to independent cloud server 330, web app 340 can search the database of active participants for a match to the uploaded unique identifier (e.g., GUID), and web app 340 can store and associate the sensor data with the participant.

In step 510, as shown in the example of FIG. 3, web app 340 can generate a report based on collected data in independent cloud server 330 and share the report with a third party. In some aspects, web app 340 can provide role-based access control for third parties (e.g., PCPs, HCPs, clinicians, research sites, institutions, industry sponsors, CROs, etc.) to review participant data, assess therapy changes, monitor participant health and safety, and/or modify parameters of the clinical trial.

In step 512, optionally, as shown in the example of FIG. 3, mobile app 320 can activate the sensor in a blinded mode such that sensor data is not displayed to the participant. In some aspects, communication between the sensor (e.g., analyte measurement system 310) and mobile app 320 can be limited to only NFC.

It is to be understood that the phraseology or terminology herein is for the purpose of description and not of limitation, such that the terminology or phraseology of the present specification is to be interpreted by those skilled in relevant art(s) in light of the teachings herein.

The following examples are illustrative, but not limiting, of the aspects of this disclosure. Other suitable modifications and adaptations of the variety of conditions and parameters normally encountered in the field, and which would be apparent to those skilled in the relevant art(s), are within the spirit and scope of the disclosure.

While specific aspects have been described above, it will be appreciated that the aspects can be practiced otherwise than as described. The description is not intended to limit the scope of the claims.

The aspects have been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific aspects will so fully reveal the general nature of the aspects that others can, by applying knowledge within the skill of the art, readily modify and/or adapt for various applications such specific aspects, without undue experimentation, without departing from the general concept of the aspects. Therefore, such adaptations and modifications are intended to be within the meaning and range of equivalents of the disclosed aspects, based on the teaching and guidance presented herein.

The breadth and scope of the aspects should not be limited by any of the above-described exemplary aspects, but should be defined only in accordance with the following claims and their equivalents.

The present invention may also be described in accordance with the following clauses:

Clause 1. A system comprising:
an analyte measurement system configured to measure an analyte of a patient, the analyte measurement system comprising an analyte sensor;
a remote device in communication with the analyte measurement system, wherein the remote device is configured to receive or retrieve sensor data from the analyte sensor; and
a processor in communication with the remote device, wherein the processor is coupled to a memory storing instructions that when executed cause the processor to:
   receive and store an order for analyte assessment associated with an electronic medical record (EMR) account of the patient;
   generate or retrieve a unique identifier associated with the order and the EMR account;
   transfer the unique identifier to the remote device;
   activate the analyte measurement system with the remote device and store the unique identifier on the analyte sensor;
   retrieve and store sensor data and the associated unique identifier from the analyte sensor;
   generate a report based on the sensor data; and
   upload the report to the EMR account associated with the unique identifier.

Clause 2. The system of clause 1, wherein the processor is configured to activate a professional blinded mode based on the unique identifier such that sensor data is not displayed to the patient.

Clause 3. The system of clause 1 or clause 2, wherein communication between the analyte measurement system and the remote device comprises near-field communication (NFC).

Clause 4. The system of any one of clauses 1 to 3, wherein the unique identifier comprises a globally unique identifier (GUID), a universally unique identifier (UUID), a unique text string, an analyte sensor serial number, patient information, an identification sticker, a quick-response (QR) code, a two-dimensional (2D) barcode, or a combination thereof.

Clause 5. The system of clause 4, wherein the unique identifier is coupled to a container configured to return the analyte sensor and/or the remote device to a location of the processor.

Clause 6. The system of clause 4, wherein the unique identifier is coupled to the analyte sensor and/or to a container storing the analyte sensor prior to use.

Clause 7. The system of any one of clauses 1 to 6, wherein the processor is located at a laboratory test facility.

Clause 8. The system of clause 7, wherein the processor is in communication with:
an order interface configured to receive the order from a third party; and
a result interface configured to provide the report to the third party.

Clause 9. The system of any one of clauses 1 to 8, wherein the remote device is configured to activate the analyte measurement system based on the unique identifier.

Clause 10. The system of clause 9, wherein the analyte measurement system is configured to store the unique identifier.

Clause 11. The system of any one of clauses 1 to 10, wherein the remote device is configured to retrieve the order based on patient information.

Clause 12. The system of clause 11, wherein the remote device is configured to:
send patient information to the processor, and
receive the unique identifier based on a match between the order and the patient information.

Clause 13. The system of any one of clauses 1 to 12, wherein the remote device comprises a second processor configured to monitor a functionality of the analyte measurement system.

Clause 14. The system of clause 13, wherein the second processor is configured to provide a notification to the patient to indicate measurement of the sensor data is complete.

Clause 15. The system of any one of clauses 1 to 14, wherein the remote device comprises a sensor management device configured to be reusable and/or disposable.

Clause 16. The system of any one of clauses 1 to 15, wherein the order is received from a clinic or a health care professional (HCP).

Clause 17. The system of any one of clauses 1 to 16, wherein the processor is in communication with a database configured to store the order.

Clause 18. The system of any one of clauses 1 to 17, wherein the processor is in communication with a data server configured to store the report.

Clause 19. The system of any one of clauses 1 to 18, wherein the analyte comprises glucose.

Clause 20. The system of any one of clauses 1 to 19, wherein the analyte comprises multiple analytes.

Clause 21. A method comprising:
receiving and storing an order for analyte assessment associated with an electronic medical record (EMR) account of a patient;
generating or retrieving a unique identifier associated with the order;
authenticating the unique identifier with a processor in communication with a remote device, wherein the remote device is configured to receive or retrieve sensor data from an analyte sensor;
measuring an analyte of the patient with an analyte measurement system in communication with the remote device, the analyte measurement system comprising the analyte sensor;
retrieving and storing sensor data from the remote device based on the order;
generating a report based on the sensor data; and
uploading the report to the EMR account associated with the unique identifier.

Clause 22. The method of clause 21, further comprising activating the analyte measurement system based on the unique identifier.

Clause 23. The method of clause 22, wherein activating comprises activating a professional blinded mode based on the unique identifier such that sensor data is not displayed to the patient.

Clause 24. The method of any one of clauses 21 to 23, further comprising communicating between the analyte measurement system and the remote device.

Clause 25. The method of clause 24, wherein communicating comprises near-field communication (NFC).

Clause 26. The method of any one of clauses 21 to 25, wherein generating the unique identifier comprises the processor generating a globally unique identifier (GUID), a universally unique identifier (UUID), a unique text string, an identification sticker, a quick-response (QR) code, a two-dimensional (2D) barcode, or a combination thereof.

Clause 27. The method of clause 26, further comprising coupling the unique identifier to a container configured to return the analyte sensor and/or the remote device to a location of the processor.

Clause 28. The method of clause 26, further comprising coupling the unique identifier to the analyte sensor and/or to a container storing the analyte sensor prior to use.

Clause 29. The method of any one of clauses 21 to 28, wherein retrieving the unique identifier comprises the processor retrieving an analyte sensor serial number, patient information, an identification sticker, a QR code, a 2D barcode, or a combination thereof.

Clause 30. The method of any one of clauses 21 to 29, wherein authenticating comprises matching patient information with the order.

Clause 31. The method of any one of clauses 21 to 30, further comprising monitoring a functionality of the analyte measurement system.

Clause 32. The method of clause 31, further comprising notifying the patient when measuring of sensor data is complete.

Clause 33. A glycemic assessment system comprising:
a glucose sensor configured to measure glucose data of a patient;
a mobile application installed on a mobile device in communication with the glucose sensor, wherein the mobile application is configured to:
   activate the glucose sensor;
   collect glucose data from the glucose sensor; and
   transmit the glucose data to a remote server; and
a web application in communication with the remote server, wherein the web application is configured to:
   store the glucose data collected from the glucose sensor;
   generate one or more reports based on the glucose data; and
   share the one or more reports with a third party.

Clause 34. The glycemic assessment system of clause 33, wherein the mobile application is configured to activate a blinded mode such that glucose data is not displayed to the patient.

Clause 35. The glycemic assessment system of clause 33 or clause 34, wherein the mobile application is configured to activate an unblinded mode such that glucose data is displayed to the patient.

Clause 36. The glycemic assessment system of any one of clauses 33 to 35, wherein communication between the glucose sensor and the mobile application comprises Bluetooth Low Energy (BLE).

Clause 37. The glycemic assessment system of any one of clauses 33 to 36, wherein the web application is configured to initiate a clinical trial.

Clause 38. The glycemic assessment system of clause 37, wherein the web application is configured to create an independent cloud server on the remote server associated with the clinical trial.

Clause 39. The glycemic assessment system of clause 37, wherein the mobile application is configured to collect the glucose data over a custom duration.

Clause 40. The glycemic assessment system of clause 39, wherein the custom duration is 14 days.

Clause 41. The glycemic assessment system of clause 37, wherein the web application is configured to provide role-based access control for users.

Clause 42. The glycemic assessment system of clause 37, wherein the web application is configured to make therapy changes during the clinical trial.

Clause 43. The glycemic assessment system of clause 37, wherein the web application is configured to monitor clinical trial metrics.

Clause 44. The glycemic assessment system of clause 43, wherein the clinical trial metrics comprise countries conducting the clinical trial, research sites conducting the clinical trial, total number of participants, number of participants per country, number of participants per research site, total usage of participants, or a combination thereof.

What is claimed is:

1. A system comprising:
an analyte measurement system configured to measure an analyte of a patient, the analyte measurement system comprising an analyte sensor;
a remote device in communication with the analyte measurement system, wherein the remote device is configured to receive or retrieve sensor data from the analyte sensor; and
a processor in communication with the remote device, wherein the processor is coupled to a memory storing instructions that when executed cause the processor to:
receive and store an order for analyte assessment associated with an electronic medical record (EMR) account of the patient;
generate or retrieve a unique identifier associated with the order and the EMR account;
transfer the unique identifier to the remote device;
activate the analyte measurement system with the remote device and store the unique identifier on the analyte sensor;
retrieve and store sensor data and the associated unique identifier from the analyte sensor;
generate a report based on the sensor data; and
upload the report to the EMR account associated with the unique identifier.

2. The system of claim 1, wherein the processor is configured to activate a professional blinded mode based on the unique identifier such that sensor data is not displayed to the patient.

3. The system of claim 1, wherein communication between the analyte measurement system and the remote device comprises near-field communication (NFC).

4. The system of claim 1, wherein the unique identifier comprises a globally unique identifier (GUID), a universally unique identifier (UUID), a unique text string, an analyte sensor serial number, patient information, an identification sticker, a quick-response (QR) code, a two-dimensional (2D) barcode, or a combination thereof.

5. The system of claim 4, wherein the unique identifier is coupled to a container configured to return the analyte sensor and/or the remote device to a location of the processor.

6. The system of claim 4, wherein the unique identifier is coupled to the analyte sensor and/or to a container storing the analyte sensor prior to use.

7. The system of claim 1, wherein the processor is located at a laboratory test facility.

8. The system of claim 7, wherein the processor is in communication with:
an order interface configured to receive the order from a third party; and
a result interface configured to provide the report to the third party.

9. The system of claim 1, wherein the remote device is configured to activate the analyte measurement system based on the unique identifier.

10. The system of claim 9, wherein the analyte measurement system is configured to store the unique identifier.

11. The system of claim 1, wherein the remote device is configured to retrieve the order based on patient information.

12. The system of claim 11, wherein the remote device is configured to:
send patient information to the processor, and
receive the unique identifier based on a match between the order and the patient information.

13. The system of claim 1, wherein the remote device comprises a second processor configured to monitor a functionality of the analyte measurement system.

14. The system of claim 13, wherein the second processor is configured to provide a notification to the patient to indicate measurement of the sensor data is complete.

15. The system of claim 1, wherein the remote device comprises a sensor management device configured to be reusable and/or disposable.

16. The system of claim 1, wherein the order is received from a clinic or a health care professional (HCP).

17. The system of claim 1, wherein the processor is in communication with a database configured to store the order.

18. The system of claim 1, wherein the processor is in communication with a data server configured to store the report.

19. The system of claim 1, wherein the analyte comprises glucose.

20. The system of claim 1, wherein the analyte comprises multiple analytes.
